(19) United States
(12) Patent Application Publication
(10) Pub. No.: US 2023/0416727 A1
(43) Pub. Date: Dec. 28, 2023

(54) HAIRPIN OLIGONUCLEOTIDES AND USES THEREOF

(71) Applicant: The University of Chicago, Chicago, IL (US)

(72) Inventors: Tao PAN, Chicago, IL (US); Christopher D. KATANSKI, Chicago, IL (US); Christopher P. WATKINS, Chicago, IL (US)

(73) Assignee: The University of Chicago, Chicago, IL (US)

(21) Appl. No.: 18/035,678

(22) PCT Filed: Nov. 5, 2021
(86) PCT No.: PCT/US2021/058258

§ 371 Date: May 5, 2023

Related U.S. Application Data
(60) Provisional application No. 63/110,605, filed on Nov. 6, 2020.

(51) Int. Cl. C12N 15/10; C12N 15/11

(52) U.S. Cl. CPC C12N 15/1065; C12N 15/11; C12N 2310/531

(57) ABSTRACT

In aspects, the invention provides a hairpin oligonucleotide comprising a 3′-terminal nucleotide, wherein the sugar component of the 3′-terminal nucleotide comprises a 2′-hydroxyl and a 3′-phosphate. In aspects, the invention provides a hairpin oligonucleotide comprising a 3′-terminal nucleotide wherein the sugar position of the 3′-terminal nucleotide comprises a 2′,3′-dialdehyde oxidation product of a sugar. In aspects, the invention provides use of a hairpin oligonucleotide in developing a biomarker. In aspects, the invention provides a solid support comprising a ligand moiety and a hairpin oligonucleotide, wherein the oligonucleotide is immobilized on the solid support through binding of the affinity moiety of the hairpin oligonucleotide to the ligand moiety of the solid support. In aspects, the invention also provides a method of preparing an RNA sequence library comprising: (a) ligating an RNA sequence to a hairpin oligonucleotide to form a construct, (b) reverse-transcribing the RNA sequence as a cDNA sequence, and (c) amplifying the cDNA sequence using PCR.

### CROSS REFERENCE TO RELATED APPLICATIONS

This patent application claims the benefit of U.S. Provisional Patent Application No. 63/110,605, filed Nov. 6, 2020, which is incorporated by reference in its entirety herein.

### STATEMENT REGARDING FEDERALLY SPONSORED RESEARCH OR DEVELOPMENT

This invention was made with government support under grant number HG008935, awarded by the National Institutes of Health. The government has certain rights in this invention.

### INCORPORATION-BY-REFERENCE OF MATERIAL SUBMITTED ELECTRONICALLY

Incorporated by reference in its entirety herein is a computer-readable nucleotide sequence listing submitted concurrently herewith and identified as follows: One 32,847Byte ASCII (Text) file named “757154_ST25.TXT,” created on Nov. 5, 2021.

### BACKGROUND OF THE INVENTION

Typical enzymatic and chemical treatments performed in RNA sequencing (RNA-seq) can present significant hurdles in sample recovery, especially for small RNAs. Additionally, due to the extremely high abundance of tRNA, small RNA-seq is often performed by separating tRNAs from other RNA by size before sequencing library construction; this separation can uncouple the data association of tRNA and other small RNAs, which may lose valuable biological information. Also, an RNA-seq procedure based on protocols that require gel purification of tRNA before and again during library construction is inefficient and requires a large amount of input material.

Most commonly used commercial RNA-seq kits are incompatible with the study of small RNAs (<about 200 nucleotides) that also contain post-transcriptional modifications. Small-RNA-seq kits often rely on sequential adaptor ligation before reverse transcription, so that abortive reverse transcription products from modifications can skew the biological information and interpretation. Conventional RNA-seq procedures and kits also lack the level of multiplexing necessary for the handling of a large number of samples.

There is a need for new RNA-seq library preparation strategies and hairpin oligonucleotides for use therewith.

### BRIEF SUMMARY OF THE INVENTION

In aspects, the invention provides a hairpin oligonucleotide comprising a 3′-terminal nucleotide, wherein the sugar component of the 3′-terminal nucleotide comprises a 2′-hydroxyl and a 3′ phosphate.

In aspects, the invention provides a hairpin oligonucleotide comprising a 3′-terminal nucleotide wherein the sugar position of the 3′-terminal nucleotide comprises a 2′, 3′-dialdehyde oxidation product of a sugar.

In aspects, the invention provides use of a hairpin oligonucleotide, the oligonucleotide comprising an affinity moiety and a 3′-terminal nucleotide, wherein the sugar component of the 3′-terminal nucleotide comprises a 2′-hydroxyl and a 3′-phosphate, in developing a biomarker.

In aspects, the invention provides a solid support comprising a ligand moiety and a hairpin oligonucleotide, the oligonucleotide comprising an affinity moiety and a 3′-terminal nucleotide, wherein the sugar component of the 3′-terminal nucleotide comprises a 2′-hydroxyl and a 3′-phosphate, and wherein the oligonucleotide is immobilized on the solid support through binding of the affinity moiety of the hairpin oligonucleotide to the ligand moiety of the solid support.

In aspects, the invention provides a method of preparing an RNA sequence library comprising: (a) ligating an RNA sequence to a hairpin oligonucleotide to form a construct, the oligonucleotide comprising a 3′-terminal nucleotide, wherein the sugar component of the 3′-terminal nucleotide comprises a 2′-hydroxyl and a 3′-phosphate, (b) reverse-transcribing the RNA sequence as a cDNA sequence, and (c) amplifying the cDNA sequence using PCR.

Additional aspects are as described herein.

### BRIEF DESCRIPTION OF THE DRAWINGS

FIG. 1 depicts RNA-sequencing (RNA-seq) library preparation, in accordance with aspects of the present invention, and shows the process undergone by oligonucleotide hairpins after an unproductive first ligation.

FIG. 2A is a schematic representation of an RNA-seq library preparation, in accordance with aspects of the invention. FIG. 2B depicts features of a capture hairpin oligo (CHO), in accordance with the invention, with embedded descriptions. FIG. 2C shows final PCR products from total RNA-seq libraries, with and without demethylase treatment. DNA size markers are indicated on the left of the gel. Major RT (reverse transcriptase) stops caused by the m1A58 and m1G37 modifications in human tRNAs are indicated on the right of the gel. TdT corresponds to the product derived from the aberrant terminal transferase activity of the RT. FIG. 2D shows final PCR products of libraries made with varying amounts of input total RNA, without and with demethylase treatment. FIG. 2E shows final PCR products of libraries starting with HEK293T total RNA (control) and human stool total nucleic acids, with and without demethylase and/or periodate treatment. FIG. 2F shows final PCR products of multiplexed oral (tongue scrape) microbiome libraries, without and with demethylase treatment.

FIG. 3A shows results of ligation of synthetic oligonucleotides to hairpin oligonucleotides. FIG. 3B shows results of reverse transcription experiments in which the ligated oligonucleotide has been immobilized on a solid support bead, with and without demethylase and/or periodate treatment. FIG. 3C shows products of PCR performed after an additional primer was added in a second ligation, showing little bias in the final product when the input RNA ends 3′-A or 3′-C. FIG. 3D demonstrates the efficiency of the dephosphorylation step. FIG. 3E shows ligation products of hairpin oligonucleotides with different terminal nucleotides, with and without periodate treatment. FIG. 3F depicts a schematic representation of measuring tRNA charging in one-pot sequencing. FIG. 3G shows final PCR products without (−,−) and with (+,+) the treatments shown in FIG. 3F.

FIG. 4A depicts RNA-seq results mapped to the E. coli genome revealing the presence of various types of RNA. FIG. 4B depicts a comparison of the relative abundance of tRNA^{Arg} or tRNA^{Leu} isoacceptors measured by sequencing or by microarray hybridization; light-colored dots on left in each pair are microarray data, dark-colored dots on right in each pair are RNA-seq data. FIG. 4C depicts a comparison of libraries made from RNA with and without demethylase treatment. FIG. 4D is a heatmap of mutation fractions along individual tRNAs. FIG. 4E depicts the abundance of non-coding RNA transcripts at rpm (reads per minute)>1, with and without demethylase.

FIG. 5A shows correlation of RNA transcript abundance among biological replicates of total RNA from E. coli grown in LB, with and without three acute stress conditions for 10 minutes. FIG. 5B shows the relationship between transcript abundance of samples treated with demethylase and untreated. FIG. 5C shows mutation rate along tRNA^{Pro}(GGG) from libraries with and without demethylase treatment. FIG. 5D shows read density along tRNA^{Pro}(GGG), with and without demethylase treatment. FIG. 5E depicts abundance of three stress-responsive small non-coding RNAs and non-responsive control RNA SRP (signal recognition particle RNA, also known as ffs) during different stresses and unstressed control. The stress-responsive sequences analyzed in FIG. 5E were: OxyS (+), responsive to oxidative stress; rhyB (triangle), responsive to iron starvation; sgrS (squares), responsive to glucose starvation; and ffs (SRP; circles), unresponsive control sequence. FIG. 5F depicts coverage density of the 3 stress-responsive small non-coding RNAs and control RNA SRP (ffs) during stresses and unstressed as control (none). FIG. 5G depicts changes in E. coli RNA abundance and modifications during stress.

FIG. 6A depicts how reads mapped to the human genome revealing RNAs of various types. FIG. 6B depicts a comparison of relative abundance of tRNA^{Arg} isoacceptors, measured by sequencing or by microarray hybridization; light-colored dots on left in each pair are microarray data, dark-colored dots on right in each pair are RNA-seq data. FIG. 6C depicts correlation of tRNA abundance results from libraries starting with 1 μg, 100 ng, or 10 ng total RNA. FIG. 6D depicts the abundance of small non-coding RNA transcripts at rpm>10.

FIG. 7A displays correlation of transcript abundance from different RNA classes with demethylase treatments. FIG. 7B displays correlation of biological replicates of different RNA classes within each class. FIG. 7C depicts correlation of tRNA abundance from demethylase-treated libraries using the inventive RNA-seq method versus a study of demethylase-treated tRNA library made using conventional methods. FIG. 7D depicts mutation rate along tRNA^{Arg}(ACG) from libraries made with and without demethylase treatment. FIG. 7E shows read density along tRNA^{Arg}(ACG) FIG. 7F depicts abundance of microRNAs detected at rpm>2. FIG. 7G depicts a read analysis of an RNA-sequencing library made from poly(A)-selected RNA. FIG. 7H shows that the majority of reads map to mRNA, with good correlations between biological replicates.

FIG. 8A depicts a schematic representation of incorporating a CMC reaction in RNA-seq. FIG. 8B depicts mutation and stop fractions at each nucleotide position in human rRNA among the biological replicates. FIG. 8C depicts mutation and stop fractions of a Ψ-rich region in 18S rRNA. FIG. 8D depicts mutation and stop fractions of a Ψ-rich region in 28S rRNA. FIG. 8E shows mutation fraction of reads at each nucleotide site along the length of the 18S rRNA. FIG. 8F shows stop fraction of reads, analyzed in the same way as in FIG. 8E.

FIG. 9A shows the assignment of reads to major RNA classes from a human tongue scraping. FIG. 9B shows the correlation of SRP RNA and 5S rRNA from various bacterial taxonomic classes. Values are computed as the Z-score of log10 abundance. FIG. 9C shows the correlation of SRP RNA abundance and the sum of all identified tRNAs for bacterial taxonomic classes, as in 9B. FIG. 9D shows the correlation of 5S rRNA and the sum of all identified tRNAs for bacterial taxonomic classes as in 9B. FIG. 9E shows reads mapping to SRP of Prevotella melaninogenica; reads map to the annotated 5′-end (top) of the gene (capitol letters), whereas the 3′-end of the transcript (bottom) 1-3 bases beyond the gene annotation into the genomic sequence (lowercase letters); extended 3′-end is consistent with the SRP structural context (middle). FIG. 9F shows reads mapping to SRP of Rothia mucilaginosa; reads map to 2-5 bases downstream of the annotated 5′-end (top) of the gene, while the 3′-end (bottom) shows heterogeneity between individuals with the 3′-end varying by 4-8 nt short of the annotated end.

FIG. 10A shows the taxonomic composition of microbes from a human tongue scraping calculated using either tRNA, 5S rRNA, SRP RNA, or measured by 16S amplicon gene sequencing. FIG. 10B shows the fold change in tongue microbe abundance between 2 sequential days, for 4 different individuals, as measured by tRNA, 5S rRNA, SRP RNA, and 16S amplicon sequencing. FIG. 10C shows read assignment to different major RNA classes from human stool. FIG. 10D shows the taxonomic composition of microbes from two human stool samples calculated using either tRNA, 5S rRNA, SRP RNA, or measured by 16S amplicon gene sequencing.

FIG. 11A shows the taxonomic composition of microbes from 4 different human tongue scrapings calculated using either tRNA, 5S rRNA, SRP RNA, or measured by 16S amplicon gene sequencing. FIG. 11B shows the taxonomic composition of microbes from a human tongue scraping calculated using tRNAs bearing either anticodon “TTT” or “CTT”.

FIG. 12A shows a heat map of mutation rates along individual tRNAs of bacteria from the genus Rothia from human tongue scraping. FIG. 12B shows a heat map as in A, but identifies mutations that are sensitive to demethylase treatment. FIG. 12C shows the mutation rate at position 37 and surrounding bases of select tRNAs from genus. FIG. 12D shows mutation rate at position 22 from select tRNAs in several bacterial taxons from human tongue with and without demethylase treatment. FIG. 12E identifies N1-methyadenosine (m1A) at position 58 (m1A58) in Actinobacteria from human tongue as in D. FIG. 12F shows the mutation rate at position 22 for select bacterial classes without demethylase treatment from 4 human tongue scrapings on 2 sequential days. FIG. 12G shows the mutation rate at position 58 for Actinobacteria without demethylase treatment from 4 human tongue scrapings on 2 sequential days. FIG. 12H identifies m1A22 in select bacteria classes as in D, from human stool. FIG. 12I identifies m1A58 in Actinobacteria as in E, from human stool.

FIG. 13 depicts a histogram of tRNAs detected in samples obtained from the noses of SARS-CoV-2 infected individuals.

FIG. 14 depicts the results of tRNA analyses from samples obtained from nasopharyngeal swabs from healthy controls and influenza- and SARS CoV-2-infected patients. FIG. 14A shows tRNA fragmentation patterns in sequential regions along the tRNA sequence for the three patient groups. FIG. 14B shows the fraction of tRNA reads in the 5′-half fragments of specific tRNAs among the three patient groups; ns, not significant, P-values : *<0.05; ** <0.01; *** <10⁻³, and **** <10⁻⁴. FIG. 14C shows the relative abundance of specific tRNAs relative to small rRNAs in the same sample among the three patient groups. FIG. 14D shows the patterns of specific tRNA base modification profiles among the samples.

FIG. 15 depicts measures of tRNA-seq abundance, modification, and fragmentation in tumor and adjacent tissues from 6 patients with colorectal cancer (CRC). FIG. 15A shows abundance of tRNA^{Ala}(TGC) is consistently higher in tumor than adjacent tissue (left panel). By contrast, tRNA^{Leu}(AAG) levels are variable, highlighting the heterogeneity of different tumors (right panel). FIG. 15B, upper panel, shows that modification in specific tRNAs can be detected by misincorporations (mutations) in sequencing. The lower panel shows that treatment of samples with demethylase enzymes can remove one type of base modification (m1A), while not affecting another type (I). FIG. 15C shows tRNA fragments produced from cellular nuclease cleavage responding to different cellular conditions.

FIG. 16 depicts tumor expression patterns of mitochondrial tRNAs in individual patients. FIG. 16A shows that expression of mitochondrial tRNAs is lower in tumor compared to adjacent tissues for 4 out of 6 patients. FIG. 16B shows that including a larger data set of mitochondrial tRNA expression data reveals that tumors from high BMI (body mass index) patients have higher mitochondrial tRNA gene expression compared to tumors from low BMI patients.

FIG. 17 depicts the composition of microbial communities measured by 5S rRNA expression in CRC patients.

FIG. 18 depicts E. faecalis tRNA^{Tyr} data from one patient, demonstrating sub-species detection. FIG. 18A shows that base misincorporation events during sequencing can be due to tRNA modifications (m1A) or to genetic diversity (SNP) in the microbiome sample. Misincorporation at position 7 reflects genetic diversity among closely related bacterial species, and FIG. 18B shows that the species composition is significantly altered after surgery. Misincorporation at position 23 reflects a base modification, and FIG. 18C shows that the fraction of this modification changes after surgery.

### DETAILED DESCRIPTION OF THE INVENTION

In aspects, the invention provides a hairpin oligonucleotide comprising a 3′-terminal nucleotide, wherein the sugar component of the 3′-terminal nucleotide comprises a 2′-hydroxyl and a 3′-phosphate. In aspects, the sugar component of the 3′-terminal nucleotide can be a pentose and the pentose can be ribose.

In aspects, the invention provides a hairpin oligonucleotide comprising a 3′-terminal nucleotide wherein the sugar position of the 3′-terminal nucleotide comprises a 2′, 3′-dialdehyde oxidation product of a sugar.

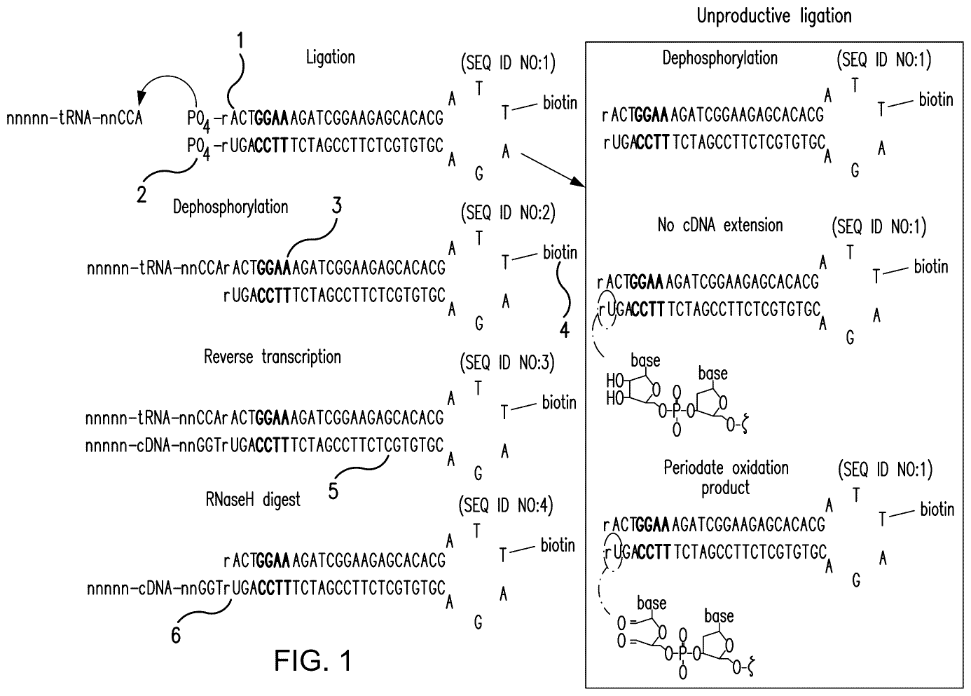

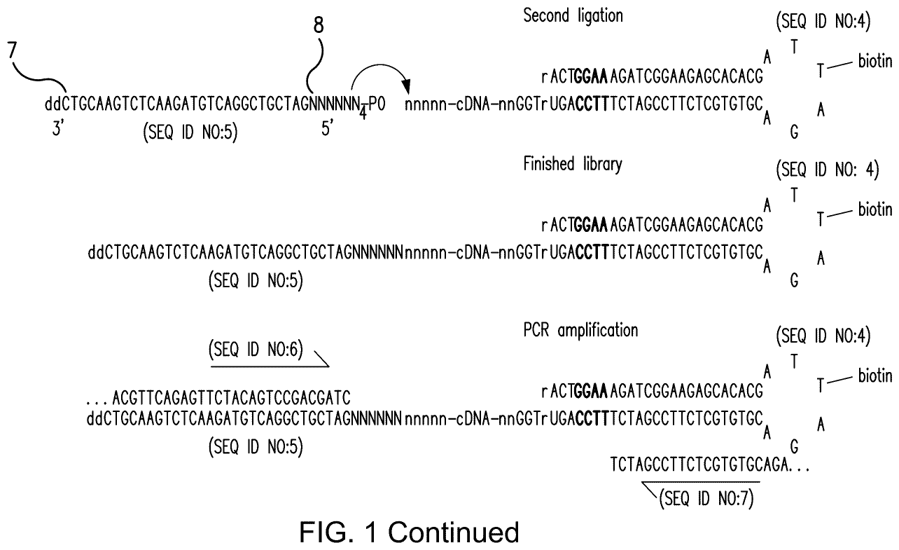

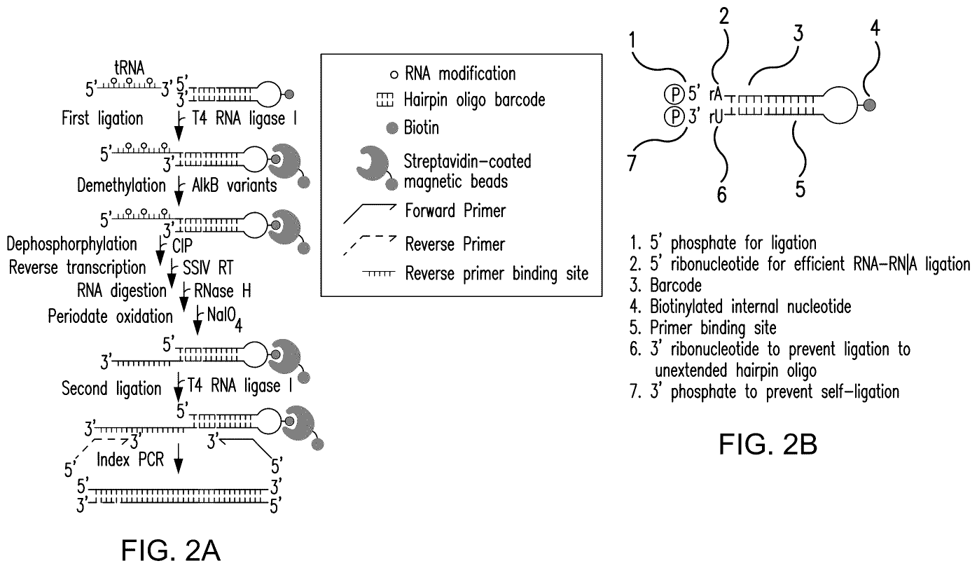

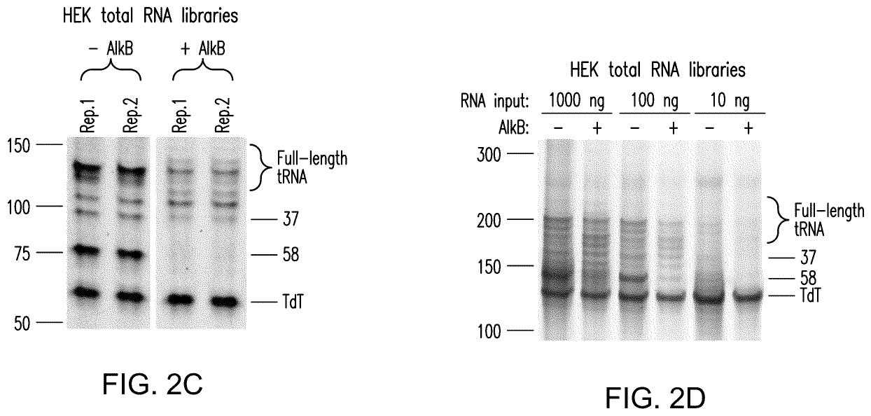

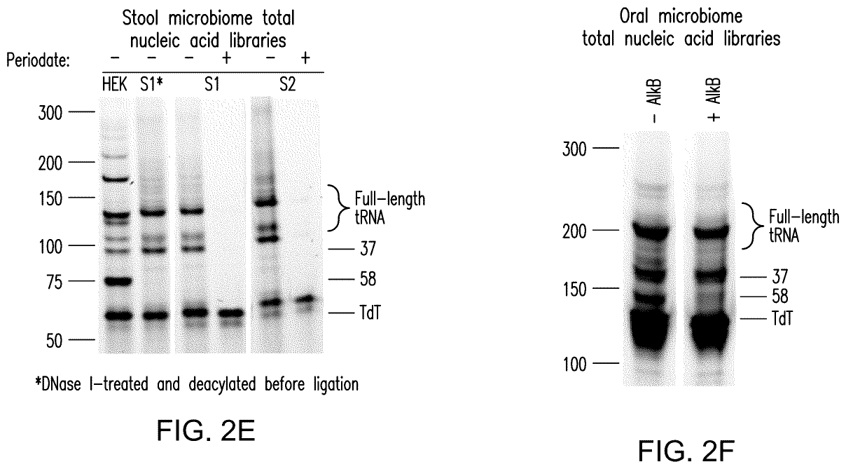

As used herein, an “oligonucleotide” is a polynucleotide chain, typically less than 200 nucleotides long, in aspects being 10 to 80 nucleotides (e.g., 10, 20, 30, 40, 50, 60, 70, or 80 nucleotides). Oligonucleotides may be single-stranded or double-stranded, and may be comprised of DNA, RNA, or both. A “hairpin oligonucleotide” refers to a type of polynucleotide having a self-complementary sequence such that the polynucleotide can fold back on itself to form a structure having a double-stranded stem with a single-stranded loop (see, e.g., FIGS. 1 and 2).

In aspects, any hairpin oligonucleotide described herein can further comprise a 5′-terminal ribonucleotide. The 5′-terminal ribonucleotide can include a 5′-phosphate.

In aspects, any hairpin oligonucleotide described herein can further comprise: (i) a barcode sequence; (ii) an affinity moiety-tagged nucleotide; and a (iii) a primer binding site. As depicted in FIG. 2B, the barcode and primer binding site sequences, in an aspect of the invention, can be embedded within the stretches of the polynucleotide sequence that form the stem region of the hairpin oligonucleotide, while the affinity moiety-tagged nucleotide, in an aspect of the invention, can be internal to the loop of the hairpin nucleotide.

In aspects, the hairpin oligonucleotide can comprise a nucleotide sequence of Formula (I): 5′-Phos-rA CT-X-AGA TCG GAA GAG CAC ACG AT (SEQ ID. NO: 86)-LT-AGA CGT GTG CTC TTC CGA TCT (SEQ ID NO: 87)-Z-AG rU-3′-Phos, wherein X is a barcode of at least 3, 4, 5 or 6 nucleotides, LT is a Thymine nucleotide tagged with an affinity moiety, and Z is a sequence of nucleotides that is the reverse complement of the barcode sequence. In aspects, a nucleotide sequence of Formula (II) can comprise: 5′-Phos-rA CT-X-GAT CGT CGG ACT GTA GAA CAT (SEQ ID NO: 88)-LT-AG AGT TCT ACA GTC CGA CGA TC (SEQ ID NO: 89)-Z-AG rU-3′-Phos, wherein X is a barcode of at least 3, 4, 5 or 6 nucleotides, LT is a Thymine nucleotide tagged with an affinity moiety, and Z is a sequence of nucleotides that is the reverse complement of the barcode sequence.

The following table presents full-length DNA sequences of the exemplary hairpin oligonucleotides described above:

TABLE 1
Exemplary Hairpin Oligonucleotides

| | SEQ | Sequence |
|---|---|---|
| | ID | |
| Description | NO: | |
| Fomula (I) | 14 | ACTNNNAGATCGGAAGAGCACACGATTAGA |
| oligo, | | CGTGTGCTCTTCCGATCTNNNAGU |
| with 3-base | | |
| barcode | | |
| Fomula (I) | 15 | ACTNNNNAGATCGGAAGAGCACACGATTAG |
| oligo, | | ACGTGTGCTCTTCCGATCTNNNNAGU |
| with 4-base | | |
| barcode | | |
| Fomula (I) | 16 | ACTNNNNNAGATCGGAAGAGCACACGATTA |
| oligo, | | GACGTGTGCTCTTCCGATCTNNNNNAGU |
| with 5-base | | |
| barcode | | |
| Fomula (I) | 17 | ACTNNNNNNAGATCGGAAGAGCACACGATT |
| oligo, | | AGACGTGTGCTCTTCCGATCTNNNNNNAGU |
| with 6-base | | |
| barcode | | |
| Fomula (II) | 18 | ACTNNNGATCGTCGGACTGTAGAACATTAG |
| oligo, | | AGTTCTACAGTCCGACGATCNNNAGU |
| with 3-base | | |
| barcode | | |
| Fomula (II) | 19 | ACTNNNNGATCGTCGGACTGTAGAACATTA |
| oligo, | | GAGTTCTACAGTCCGACGATCNNNNAGU |
| with 4-base | | |
| barcode | | |
| Fomula (II) | 20 | ACTNNNNNGATCGTCGGACTGTAGAACATT |
| oligo, | | AGAGTTCTACAGTCCGACGATCNNNNNAGU |
| with 5-base | | |
| barcode | | |
| Fomula (II) | 21 | ACTNNNNNNGATCGTCGGACTGTAGAACAT |
| oligo, | | TAGAGTTCTACAGTCCGACGATCNNNNNNA |
| with 6-base | | GU |
| barcode | | |

As used herein, the term “barcode” refers to a known nucleic acid sequence that allows some feature of a polynucleotide with which the barcode is associated to be identified. Often, the feature of the polynucleotide to be identified is the sample from which the polynucleotide is derived. In aspects, barcodes are at least 3, 4, 5, 6 or more nucleotides in length. In aspects, barcodes are not shorter than 3 nucleotides in length. In aspects, each barcode in a mixture containing a plurality of barcodes differs from every other barcode in the plurality by at least two nucleotide positions, such as at least 2, 3, 4, 5, or more positions. Preferably, the barcodes in a mixture differ from each other by at least three nucleotide positions. In general, barcodes are of sufficient length and comprise sequences that are sufficiently different to allow the identification of samples based on the barcodes with which they are associated.

As used herein, the term “primer” refers to a nucleotide sequence capable of hybridizing with a complementary nucleotide sequence and capable of providing a starting point for DNA synthesis. Primers are of sufficient length to provide specific binding to their complementary nucleotide sequence. Primers can be of 6, 7, 8, 9, 10 or more bases in length, typically of 15, 16, 17, 18, 19, or 20 nucleotides in length. A primer can be, for example, a sequence within a longer single-stranded polynucleotide sequence. Alternatively, a primer can be a single-stranded oligonucleotide.

In aspects, any hairpin oligonucleotide described herein can be immobilized on a solid support. The solid support may be any solid support suitable for use in biochemical processes, such as column chromatography. For example, the solid support may be a controlled-pore glass, or a polymeric support such as a polystyrene support. Suitable solid supports are often polymeric and may have a variety of forms and compositions. Some solid supports derive from naturally occurring materials, and others from naturally occurring materials that have been synthetically modified, and others are synthetic materials. Examples of suitable support materials include, but are not limited to, polysaccharides such as agarose and dextran, polyacrylamides, polystyrenes, polyvinyl alcohols, copolymers of hydroxyethyl methacrylate and methyl methacrylate, silicas, teflons, glasses, and the like. In aspects, the solid support may comprise beads. In aspects, the beads may be substantially uniform spherical beads.

In aspects, any hairpin oligonucleotide described herein can be used in preparing an RNA-sequence library. In aspects, the hairpin oligonucleotide is used in a multiplex method of preparing an RNA-sequence library. As used herein, the term “multiplexing” refers to pooling a large number of samples and subjecting the pooled samples to one or more biochemical processes simultaneously. Exemplary methods are described below.

In aspects, the invention provides a solid support comprising a ligand moiety and a hairpin oligonucleotide, the oligonucleotide comprising an affinity moiety and a 3′-terminal nucleotide, wherein the sugar component of the 3′-terminal nucleotide comprises a 2′-hydroxyl and a 3′-phosphate, and wherein the oligonucleotide is immobilized on the solid support through binding of the affinity moiety of the hairpin oligonucleotide to the ligand moiety of the solid support.

The affinity moiety on the oligonucleotide and the ligand moiety on the solid support form an affinity pair. An “affinity pair” comprises an affinity moiety and a ligand moiety that specifically bind each other, e.g., through an intrinsic property such as hydrophobicity, hydrophilicity, hydrogen bonds, polarity, charges, fluorophilicity, etc. The terms “affinity moiety” and “ligand moiety” identify the moieties as capable of forming an affinity pair without limiting the identities of the moieties themselves (e.g., the ligand moiety need not be smaller than the affinity moiety). One well-known type of affinity pair is a protein and its ligand. The affinity moiety and the ligand moiety can each be attached separately to the oligonucleotide and the solid support through an orthoester linker, either directly or indirectly. In aspects, the affinity moiety is a biotin tag, a maltose tag, glutathione tag, an adamantane tag, an arylboronic acid tag, poly-histidine peptide tag, poly-sulfhydryl tag, a maleimide tag, an azido tag, and the like. In these aspects, the corresponding ligand moiety is avidin or streptavidin, maltose binding protein, glutathione S-tranferase (GST), a cucurbituril or cyclodextrin, a diol containing molecule, an immobilized metal affinity chromatography (IMAC) matrix, a sulfhydryl-containing compound, an alkyne or cyclooctyne, and the like. In aspects, the affinity moiety can be biotin and the ligand moiety can be streptavidin (see, e.g., FIGS. 2A-B). A skilled person can decide which member of the affinity pair to attach to the oligonucleotide and which to attach the solid support. As described above, in aspects, the solid support can be a bead. In aspects, the beads may be substantially uniform spherical beads.

The solid support may comprise any hairpin oligonucleotide as described herein. For example, the oligonucleotide may further comprise (a) a 5′-terminal nucleotide as a ribonucleotide, (b) a barcode sequence, (c) a nucleotide tagged with the affinity moiety internal to the loop of the hairpin, and (d) a primer binding site.

In aspects, the invention provides a method of preparing an RNA sequence library comprising:
(a) ligating an RNA sequence to a hairpin oligonucleotide to form a construct, the oligonucleotide comprising a 3′-terminal nucleotide, wherein the sugar component of the 3′-terminal nucleotide comprises a 2′-hydroxyl and a 3′-phosphate,
(b) reverse-transcribing the RNA sequence as a cDNA sequence, and
(c) amplifying the cDNA sequence using PCR.

In aspects, the method can include a hairpin oligonucleotide further comprising: (i) a 5′-terminal nucleotide as a ribonucleotide, (ii) a barcode sequence, (iii) an affinity moiety-tagged nucleotide internal to the loop of the hairpin, and (iv) a primer binding site.

FIG. 2A schematically depicts a non-limiting aspect of a hairpin oligonucleotide of the invention used in the preparation of a RNA-seq library. The process can begin with the ligation of the prepared capture hairpin oligonucleotide (CHO) to an RNA molecule, wherein the CHO comprises a 3′-terminal nucleotide, wherein the sugar component of the 3′-terminal nucleotide comprises a 2′-hydroxyl and a 3′-phosphate. A hairpin oligonucleotide can be designed to enable “on-bead” RNA-sequencing library preparation. As an exemplary CHO, the features of the CHO as depicted in FIG. 2B are: (1) a 5′-phosphate for efficient ligation; (2) a 5′-terminal ribonucleotide for efficient RNA-RNA ligation; (3) a barcode sequence to enable multiplexing/mixing of multiple samples; (4) an affinity-moiety tagged nucleotide internal to the loop of the CHO to enable immobilization; (5) a primer binding site; (6) a 3′-terminal nucleotide, wherein the sugar component of the 3′-terminal nucleotide comprises a 2′-hydroxyl to prevent ligation to unextended hairpin oligonucleotide and to enable oxidation of unproductive ligation product; and (7) a 3′-phosphate to prevent self-ligation of the CHO. In aspects, the sugar component of the 3′-terminal nucleotide can be a pentose and the pentose can be ribose.

The RNA molecule may be any suitable RNA sequence. In aspects, the RNA sequence can comprise total RNA (e.g., several different constructs formed by ligation of hairpin oligonucleotide to the different types of RNA in a sample). In another aspect, the RNA sequence can be small RNA. Small RNAs include tRNAs, microRNAs, piRNAs, fragments of tRNAs, rRNAs, long non-coding RNAs (lncRNAs), spliceosomal RNAs (snRNAs), small nucleolar RNAs (snoRNAs), and others. In aspects, the RNA sequence used can be tRNA.

As an aspect, FIGS. 1 and 2A show ligation of a barcode-bearing CHO to a tRNA with RNA ligase. Any suitable RNA ligase may be used, for example T4 RNA ligase 1 or 2, or the like. In aspects, the ligase used can be T4 RNA ligase 1. The 5′-terminal ribonucleotide can include a 5′-phosphate and promote ligation efficiency. The 3′-phosphate blocks self-ligation of the hairpin oligonucleotide in the first ligation, which improves the efficiency of the hairpin oligonucleotide ligation to the RNA.

After the barcode ligation reaction, all subsequent reactions can be performed after immoblization of the tRNA-bearing CHO on a solid support. The oligonucleotide can be immobilized on the solid support through binding of the affinity moiety of the hairpin oligonucleotide to the ligand moiety of the solid support. This facilitates the removal of excess reagents in every step with simple washes, significantly reducing sample loss during each step.

In aspects of the method, the solid support comprises a ligand moiety and a hairpin oligonucleotide, the oligonucleotide comprising an affinity moiety and a 3′-terminal nucleotide, wherein the sugar component of the 3′-terminal nucleotide comprises a 2′-hydroxyl and a 3′-phosphate, and wherein the oligonucleotide is immobilized on the solid support through binding of the affinity moiety of the hairpin oligonucleotide to the ligand moiety of the solid support. In aspects, the affinity moiety can be biotin and the ligand moiety can be streptavidin (see, e.g., FIGS. 2A-B).

In aspects of the method, the solid support can be a bead. In aspects, the solid support immobilizes an oligonucleotide which further comprises: (a) a 5′-terminal nucleotide as a ribonucleotide, (b) a barcode sequence, (c) a nucleotide tagged with the affinity moiety internal to the loop of the hairpin, and (d) a primer binding site. In aspects, the solid support can be used in preparing an RNA-sequence library. In other aspects, the solid support can be used in a multiplex method of preparing an RNA-sequence library. After barcoded CHO is immobilized on the solid support, the samples can be pooled, which allows for multiplexing.

After binding of the tRNA-bearing CHO to the solid support, optional enzymatic or chemical treatments of the RNA can be performed to profile RNA modifications or map RNA structures. For example, demethylase treatment improves the efficiency and quantitation in tRNA and tRNA fragment sequencing, and provides validation for discovering new RNA modifications such as N1-methyladenosine (m1A) in the microbiome tRNA or in mRNA. Many RNA structural mappings involve chemical reaction such as using 2-methylnicotinic acid imidazolide for 2′-OH (SHAPE) or dimethylsulfate/kethoxal for base conformation. In RNA modification studies, chemical reactions are used in the identification of pseudouridine (Ψ) or 5-methylcytosine (m5C) sites. For example, FIG. 2A depicts treatment of the bead-immobilized CHO comprising tRNA with a demethylase to remove Watson-Crick face methylations in the tRNA. In aspects, the demethylase can be an AlkB demethylase mixture.

FIGS. 1 and 2A further depict examples of other procedures that can be used in preparation of an RNA-seq library. Following demethylation, the 3′-phosphate group can be removed with alkaline phosphatase. In aspects, the alkaline phosphatase is from calf intestine (CIP).

After dephosphorylation, the 3′-OH of the CHO can be extended by reverse transcriptase to make a cDNA copy of the RNA. Any suitable reverse transcriptase (RT) can be used, for example, TGI RT, AMV RT, ThermoScript™ RT (Invitrogen™), MMLV RT, SuperScript™ IV RT (Invitrogen™) and the like. In aspects, the reverse transcriptase can be SuperScript™ IV RT (Invitrogen™).

After reverse transcription, the tRNA sequence can be digested with an RNase. An endonuclease RNase capable of degrading the RNA strand in a DNA/RNA duplex is desired, such as RNase H. In aspects, the RNase can be RNase H.

After RNase digestion, the CHO can be oxidized with periodate, preferably with sodium periodate (NaIO₄). As illustrated in FIG. 1, the CHO can have different fates after the initial ligation step, such that only some of the CHO will be susceptible to oxidation when treated with periodate.

All CHO can undergo dephosphorylation, but the end products of the dephosphorylation can be different. CHO that were successfully ligated to an RNA will have undergone chain extension with reverse transcriptase from the 3′-OH. These CHO will then have a 3′-terminal deoxyribonucleotide, that is, they will have a terminal 3′-OH and 2′-H. These CHO will not have the 2′,3′-diol structure necessary for periodate oxidation. Thus, only CHO terminating in both 2′- and 3′-OH, that is, CHO that did not undergo ligation and so have not been extended with cDNA, will be oxidized by periodate (see, e.g., FIG. 1B). The terminal dialdehyde of the oxidized CHO (see, e.g., FIG. 1B) will prevent these CHO from undergoing the following second ligation, and so will reduce technical noise in downstream reactions.

A second ligation can follow (see, e.g., FIGS. 1 and 2A), adding a second “reverse” primer binding site before PCR amplification so that both complementary DNA strands will be produced during PCR. The second ligation oligonucleotide can include a Unimolecular Index (UMI) sequence at the 5′-end and a dideoxy nucleotide at the 3′-end (see, e.g., FIG. 1). The 3′-terminal dideoxy nucleotide blocks self-ligation of the oligonucleotide. UMIs are short sequences used to uniquely tag each molecule in a sample library. They are a type of molecular barcoding that provides error correction and increased accuracy during sequencing and enables deduplication of PCR artifacts during RNA-sequencing. Exemplary oligonucleotides for use in the second ligation step are an oligonucleotide of Formula (III): 5′-Phos-NNN NNN GAT CGT CGG ACT GTA GAA-3ddC (SEQ ID NO: 22) and an oligonucleotide of Formula (IV): 5′-Phos-NNN NNN AGA TCG GAA GAG CAC ACG-3ddC (SEQ ID NO: 23), wherein the strings of Ns represent UMI sequences of 6 nucleotides in length.

After RNA-seq library preparation, the cDNA extended-CHO can undergo PCR amplification. Any suitable PCR reagent system and thermocycler instrument may be used for PCR. The PCR products are free in solution and can readily be used for DNA sequencing.

As illustrated above, the method may include several aspects. In aspects, the method can further comprise dephosphorylating the 3′-phosphate after ligation, and oxidizing 3′-terminal nucleotides comprising a 2′,3′-diol with periodate after reverse transcription. In aspects, the method can also comprise demethylating Watson-Crick face methylations on nucleotides of the RNA sequence after ligation and before dephosphorylation. In aspects, the method can also comprise digesting the RNA sequence after reverse transcription and performing a second ligation to add a second primer binding site before amplification. In aspects, the method can further comprise immobilizing the construct on a solid support after the first ligation. In aspects, the method can also comprise dephosphorylating the 3′-phosphate after immobilization and oxidizing 3′-terminal nucleotides comprising a 2′,3′-diol with periodate after reverse transcription. In aspects, the method can also comprise demethylating Watson-Crick face methylations on nucleotides of the RNA sequence after immobilization and before dephosphorylation. In aspects, the method can also comprise digesting the RNA sequence after reverse transcription and performing a second ligation to add a second primer binding site before amplification. In aspects, the method can use RNA comprising total RNA, small RNAs, tRNAs, micro RNAs, piRNAs, or any combination thereof. In aspects, the method can comprise a multiplex method. In aspects, the present invention can involve an affinity moiety-tagged oligonucleotide that is used for the barcode adapter ligation, immobilization, and reverse transcription, followed by second adapter ligation, and on-bead PCR. The unification of multiple steps in RNA-seq library construction enables multiplexing of many samples in the same reaction, thus reducing time, reagents, and technical noise, and greatly increasing throughput. The design also allows for inclusion of efficient enzymatic and chemical treatment of RNA on-bead.

Development of a solid support-based RNA-seq method enables multiplexed sequencing library preparation, on-bead enzymatic and chemical treatment, one-pot tRNA abundance, modification and charging measurement, and analysis of total nucleic acid microbiome samples without the interference of DNA.

The advantage of being able to carry out most of the procedures in sequencing library construction on a solid support is that it allows for rapid exchange of buffers and reagents between each procedure, thorough removal of contaminants, and elimination of all procedures that require size selection or adaptor/RT primer removal. The solid support platform also allows for on-bead treatment of RNA with enzymes, such as demethylases used to remove Watson-Crick face methylations in RNA, enabling efficient and quantitative tRNA sequencing and validation of microbiome tRNA modification.

In aspects, the inventive hairpin oligonucleotides can be used in developing a biomarker. In aspects, developing the biomarker comprises generating a tRNA fragmentation profile. In aspects, the biomarker can be developed from solid biopsy or from liquid biopsy. In aspects, the biomarker can be developed from liquid biopsy. The term “liquid biopsy,” also known as fluid biopsy or fluid phase biopsy, refers to sampling and analysis of non-solid biological material, such as material collected from blood, plasma, saliva, urine, nasal secretions, etc. In aspects, the biomarker can be a biomarker for viral disease severity or for cancer.

The inventive hairpin oligonucleotides, total RNAs, cDNAs, primers, nucleic acids, proteins, polypeptides and cells referred to herein (including populations thereof), can be isolated and/or purified. The term “isolated,” as used herein, means having been removed from its natural environment. The term “purified,” as used herein, means having been increased in purity, wherein “purity” is a relative term, and not to be necessarily construed as absolute purity. For example, the purity can be at least about 50%, can be greater than about 60%, about 70%, about 80%, about 90%, about 95%, or can be about 100%.

The following includes certain aspects of the invention.
1. A hairpin oligonucleotide comprising a 3′-terminal nucleotide, wherein the sugar component of the 3′-terminal nucleotide comprises a 2′-hydroxyl and a 3′-phosphate.
2. The hairpin oligonucleotide of aspect 1, wherein the sugar component of the 3′-terminal nucleotide is a pentose and the pentose is ribose.
3. A hairpin oligonucleotide comprising a 3′-terminal nucleotide wherein the sugar position of the 3′-terminal nucleotide comprises a 2′,3′-dialdehyde oxidation product of a sugar.
4. The hairpin oligonucleotide of any one of aspects 1-3, further comprising a 5′-terminal ribonucleotide.
5. The hairpin oligonucleotide of any one of aspects 1-4, further comprising:
   (a) a barcode sequence,
   (b) an affinity moiety tagged-nucleotide internal to the loop of the hairpin, and
   (c) a primer binding site.
6. The hairpin nucleotide of aspect 5, comprising the sequence:

5′-Phos-rACT-X-AGA TCG GAA GAG CAC ACG AT
(SEQ ID NO: 86)-LT-AGA CGT GTG CTC TTC CGA
TCT (SEQ ID NO: 87)-Z-AG rU-3′-Phos

wherein X is a barcode of at least 3, 4, 5 or 6 nucleotides, LT is an affinity moiety tagged-Thymine nucleotide, and Z is a sequence of nucleotides that is the reverse complement of the barcode sequence.
7. The hairpin nucleotide of aspect 5, comprising the sequence:

5′-Phos-rACT-X-GAT CGT CGG ACT GTA GAA CAT
(SEQ ID NO: 88)-LT-AG AGT TCT ACA GTC CGA
CGA TC (SEQ ID NO: 89)-Z-AG rU-3′-Phos,

wherein X is a barcode of at least 3, 4, 5 or 6 nucleotides, LT is an affinity moiety tagged-Thymine nucleotide, and Z is a sequence of nucleotides that is the reverse complement of the barcode sequence.
8. The hairpin oligonucleotide of any one of aspects 1-7 immobilized on a solid support.
9. Use of the hairpin oligonucleotide of any one of aspects 1-8 in preparing an RNA-sequence library.
10. Use of the hairpin oligonucleotide of any one of aspects 1-8 in a multiplex method of preparing an RNA-sequence library.
11. Use of the hairpin oligonucleotide of any one of aspects 1-8 in developing a biomarker.
12. The use of aspect 11, wherein the biomarker is developed from liquid biopsy.
13. The use of aspect 11 or 12, wherein developing the biomarker comprises generating a tRNA fragmentation profile.
14. The use of the hairpin oligonucleotide of any one of aspects 1-8 in developing a biomarker for viral disease severity.
15. Use of the hairpin oligonucleotide of any one of aspects 1-8 in developing a biomarker for cancer.
16. A solid support comprising a ligand moiety and a hairpin oligonucleotide, the oligonucleotide comprising an affinity moiety and a 3′-terminal nucleotide, wherein the sugar component of the 3′-terminal nucleotide comprises a 2′-hydroxyl and a 3′-phosphate, and wherein the oligonucleotide is immobilized on the solid support through binding of the affinity moiety of the hairpin oligonucleotide to the ligand moiety of the solid support.
17. The solid support of aspect 16, wherein the affinity moiety is biotin and the ligand moiety is streptavidin.
18. The solid support of aspect 16 or 17, wherein the solid support is a bead.
19. The solid support of any one of aspects 16-18, wherein the oligonucleotide further comprises:
   (a) a 5′-terminal nucleotide as a ribonucleotide,
   (b) a barcode sequence,
   (c) a nucleotide tagged with the affinity moiety internal to the loop of the hairpin, and
   (d) a primer binding site.
20. Use of the solid support of any one of aspects 16-19 in preparing an RNA-sequence library.
21. Use of the solid support of any one of aspects 16-19 in a multiplex method of preparing an RNA-sequence library.
22. A method of preparing an RNA sequence library comprising:
   (a) ligating an RNA sequence to a hairpin oligonucleotide to form a construct, the oligonucleotide comprising a 3′-terminal nucleotide, wherein the sugar component of the 3′-terminal nucleotide comprises a 2′-hydroxyl and a 3′-phosphate,
   (b) reverse-transcribing the RNA sequence as a cDNA sequence, and
   (c) amplifying the cDNA sequence using PCR.
23. The method of aspect 22, wherein the hairpin oligonucleotide further comprises:
   (i) a 5′-terminal nucleotide as a ribonucleotide,
   (ii) a barcode sequence,
   (iii) an affinity moiety-tagged nucleotide internal to the loop of the hairpin, and
   (iv) a primer binding site.
24. The method of aspect 22 or aspect 23, further comprising dephosphorylating the 3′-phosphate after ligation and oxidizing 3′-terminal nucleotides comprising a 2′,3′-diol with periodate after reverse transcription.
25. The method of aspect 24, further comprising demethylating Watson-Crick face methylations on nucleotides of the RNA sequence after ligation and before dephosphorylation.
26. The method of any one of aspects 22-25, further comprising digesting the RNA sequence after reverse transcription and performing a second ligation to add a second primer binding site before amplification.
27. The method of aspect 22 or aspect 23, further comprising immobilizing the construct on a solid support after ligation.
28. The method of aspect 27, further comprising dephosphorylating the 3′-phosphate after immobilization and oxidizing 3′-terminal nucleotides comprising a 2′,3′-diol with periodate after reverse transcription.
29. The method of aspect 28, further comprising demethylating Watson-Crick face methylations on nucleotides of the RNA sequence after immobilization and before dephosphorylation.
30. The method of any one of aspects 27-29, further comprising digesting the RNA sequence after reverse transcription and performing a second ligation to add a second primer binding site before amplification.
31. The method of any one of aspects 22-30, wherein the RNA sequence comprises total RNA, small RNAs, tRNAs, micro RNAs, piRNAs, or any combination thereof.
32. The method of any one of aspects 22-31, wherein the method comprises a multiplex method.

It shall be noted that the preceding are merely examples of aspects. Other exemplary aspects are apparent from the entirety of the description herein. It will also be understood by one of ordinary skill in the art that each of these aspects may be used in various combinations with the other aspects provided herein.

The following examples further illustrate the invention but, of course, should not be construed as in any way limiting its scope.

### EXAMPLE 1

### Methods

The following methods were used in RNA-seq library preparation, in accordance with aspects of the invention.

### Preparation of the RNA

tRNA Deacylation

Total RNA was prepared for library construction by first deacylating in a solution of 100 mM TrisHCl, pH 9.0 at 37° C. for 30 minutes, then neutralizing by addition of sodium acetate, pH 4.8 at a final concentration of 180 mM. Deacylated RNA was then ethanol precipitated and resuspended in water, or desalted using a Zymo Oligo Clean-and-Concentrator™ spin column.

One-Pot Deacylation and β-Elimination for tRNA Charging

Up to 500 ng of total RNA in 7 μL was used for optional one-pot beta-elimination prior to library construction. To start, 1 μL of 90 mM sodium acetate buffer, pH 4.8 was added to 7 μL input RNA. Next, 1 μL of freshly prepared 150 mM sodium periodate solution was added and mixed; reaction conditions were 16 mM NaIO₄, 10 mM NaOAc, pH 4.8. Periodate oxidation proceeded for 30 min at room temperature. Oxidation was quenched with addition of 1 μL of 0.6 M ribose at 60 mM final and incubated for 5 minutes. Next 5 μL of freshly prepared 100 mM sodium tetraborate, pH 9.5 was added for a final concentration of 33 mM. This reaction was incubated for 30 min at 45° C. To stop β-elimination and 3′-end repair, 5 μL of T4 PNK mix (200 mM TrisHCl pH 6.8, 40 mM MgCl₂, 4 U/μL T4 PNK from New England Biolabs (NEB) was added to the reaction, and incubated at 37° C. for 20 min. T4 PNK was then heat inactivated by incubating at 65° C. for 10 min. This reaction mixture at a total of 20 μL can be used directly in the first bar-code ligation by adding 30 μL of a ligation master mix described below.

### General Protocol for RNA-Seq

### First Bar-Code Ligation

Input material were either deacylated or had undergone beta-elimination and end repair as described above. Up to 1 μg of total RNA input was used in a ligation reaction of 50 μL with the following components: 1 U/μL T4 RNA ligase I (NEB), 1×NEB T4 RNA ligase I buffer, 15% PEG 8000, 50 μM ATP, 1 mM hexaamine cobalt chloride, and 5% DMSO. After adding the ligation mix to the sample, the hairpin was added to a final concentration of 1 μM and the samples were incubated at 16° C. overnight (12+hours).

### Binding to Dynabeads

The ligation mixture was diluted by adding an equal volume of water to reduce the viscosity of the solution. Next, streptavidin-coated Dynabeads™ MyOne™ C1 (ThermoFisher) were added to each sample in a 1.2:1 excess over hairpin oligo (for example, a 50 μL reaction had 50 pmol hairpin oligo; beads were supplied at 10 mg/ml and had binding capacity of 500 pmol biotinylated oligo per mg, so 12 μL slurry were added). The bead-sample mixture was incubated at room temperature for 15 minutes. After binding, supernatants were removed, and the beads washed once with high salt wash buffer (1 M NaCl, 20 mM TrisHCl, pH 7.4) and once with low salt wash buffer (100 mM NaCl, 20 mM TrisHCl, pH 7.4).

After washing, multiple individually barcoded samples can be combined for downstream steps. At this stage, enzymatic or chemical treatments can be incorporated to the library preparation protocol such as AllcB demethylase reaction or CMC treatments (see methods below).

### Dephosphorylation

The dephosphorylation mix of 50 μL containing the following was added to the multiplexed sample on-bead: 0.04 U/μL calf intestine phosphatase (Roche), 10 mM MgCl₂, 0.5 mM ZnCl₂, 20 mM HEPES, pH 7.3. The sample was incubated at 37° C. for 30 minutes. The sample was then washed once with high salt wash buffer and once with low salt wash buffer, then resuspended in 20 μL water.

### Reverse Transcription

SuperScript™ IV VILO 5×master mix (ThermoFisher) of 5 μL was added to the dephosphorylated sample to a final volume of 25 μL and then incubated at 55° C. for 10 minutes. The sample was then washed once with high salt wash buffer and once with low salt wash buffer.

### RNase H Digestion

Beads were resuspended in the RNase H master mix of 50 μL containing 0.4 U/uL RNase H (NEB) and 1×NEB RNase H buffer and incubated at 37° C. for 15 minutes. The sample was then washed once with high salt wash buffer and once with low salt wash buffer. The sample was then resuspended in 40 μL water.

### Periodate Oxidation

A 5×solution of 250 mM sodium periodate of 10 μL in freshly prepared 0.5 M sodium acetate, pH 5 was added to the RNase H-digested sample and incubated at room temperature for 30 minutes. Afterwards, ribose was added to a final concentration of 167 mM to quench excess periodate at room temperature for 5 minutes. The sample was then washed once with high salt wash buffer and once with low salt wash buffer.

### Second Ligation

Beads were resuspended in a ligation master mix of 50 μL with the following components: 2U/μL T4 RNA ligase I (NEB), 1×NEB T4 RNA ligase I buffer, 2 μM second ligation oligo, 25% PEG 8000, 50 μM ATP, 7.5% DMSO, and 1 mM hexaamine cobalt chloride. The reaction was incubated at room temperature overnight (12+ hours). The reaction was then diluted with one volume of water to reduce viscosity, washed once with high salt wash buffer and once with low salt wash buffer, and then resuspended in water with beads at ˜10-20 mg/mL (6-12 μL per initial ligation reaction). Samples can be stored at 4° C. or frozen at −20° C.; although freezing may damage the beads, but it can still be used for the next PCR step.

### PCR

A 50 μL PCR reaction was run using 5-10% of the bead slurry products from the second ligation reaction using Q5 DNA polymerase (NEB) and following the manufacturer's instructions: 0.02 U/μL Q5 DNA polymerase, 1×Q5 reaction buffer, 0.2 mM dNTPs, 0.5 μM Illumina index primer, and 0.5 μM Illumina multiplex primer. Typical PCR cycles were 9, 12, and 15 cycles at 10 seconds at 98° C., 15 seconds at 55° C., and 72° C. for 20 seconds and then the best condition was selected. PCR reactions were then processed by DNA Clean and Concentrate kit (Zymo).

### TBE-PAGE Gel Extraction

Following desalting, PCR products were run on 10% non-denaturing TBE gels with dsDNA size markers; lanes were cut according to the desired product size, mashed by pipette tip, and then resuspended in crush-and-soak buffer (500 mM sodium acetate, pH 5.0). The gel fragments were extracted overnight and then ethanol precipitated.

### Oligonucleotide Sequences

Oligonucleotide sequences used in experiments described herein are found in the following tables.

Tables 1-3 provide exemplary hairpin oligonucleotides according to the invention. The sequences are annotated in a format compatible with ordering from Integrated DNA Technology, Inc. (IDT). For example, “/5Phos/” indicates a 5′-phosphate. The short oligonucleotide sequence (L2) listed in the last row of each table is the oligonucleotide used in conjunction with the hairpin oligonucleotide sequences listed earlier in the table in the second ligation step of the RNA-seq method. The UMI in each L2 is represented by the “N” residues; the UMIs are hexN (6 nucleotides long) to maximize sample complexity. Data shown herein resulting from use of a particular oligonucleotide in RNA-seq is identified by the Figure number.

The oligonucleotides are designed to be used in either paired-end or single-end DNA sequencing-by-synthesis methods. In paired-end sequencing, sequencing is done from both ends of a DNA fragment. A first primer is annealed and every subsequent base is determined as it is added to the growing strand. This is “read 1” sequencing of the forward strand. Next, another primer containing the UMI sequence is annealed and extended in the “indexing read” which measures the index. Lastly, a third primer is annealed and extended, which sequences the reverse strand as “read 2.” By contrast, in single-end sequencing, only read 1 and the indexing read are performed. A variety of DNA sequencing instruments and platforms are commercially available. A preferred system for performing DNA sequencing is the NGS (Next Generation Sequencing) System of Illumina, Inc.

Two types of hairpin oligonucleotides have been designed, one in which the barcode is read at the start of “read 1” sequencing, and the other in which the barcode is read at the start of “read 2” sequencing. In general, the design with the barcode at the start of “read 2” is preferred since this maximizes the “complexity” or measured sequence diversity at the beginning of the run. A sequence for the hairpin oligonucleotides designed for read 1 sequencing is /5Phos/rA CT XXXX GAT CGT CGG ACT GTA GAA CAT/iBiodT/AG AGT TCT ACA GTC CGA CGA TC ZZZZ AG rU/3Phos/ (SEQ ID NO: 19), where “X” is the barcode sequence (which is at least 3 nucleotides long; 4 nucleotide barcode shown here) and “Z” is the sequence that is the reverse complement of the “X” barcode nucleotides. A sequence for the hairpin oligonucleotides designed for read 2 sequencing is /5Phos/rA CT XXXX AGA TCG GAA GAG CAC ACG AT/iBiodT/AGA CGT GTG CTC TTC CGA TCT ZZZZ AG rU/3Phos/ (SEQ ID NO:15), where “X” is the barcode sequence (which is at least 3 nucleotides long; 4 nucleotide barcode shown here) and “Z” is the sequence that is the reverse complement of the “X” barcode nucleotides. The corresponding L2 oligonucleotides used in the indexing reads are shown in the last rows of Tables 1-3. The “read 1” design is compatible with either paired-end or single-end sequencing, as the barcode sequence will still be measured. In this form extra care can be taken with regard to complexity, which can be bolstered by using multiple barcodes, or with spike-in controls as recommended by Illumina (e.g. Phi-X control DNA).

When comparing any two sequences of the same length, the Hamming distance is the number of sequence positions at which the corresponding symbols differ. A Hamming distance for barcodes is chosen so that, if the sequencer makes an error while reading the barcode, a single error can be identified and the correct barcode can be assigned. For example, if a Hamming distance were 1, then a single error would turn one barcode into another, and the error would never be detected. With a Hamming distance of 2, a single error can be detected, but the erroneous read could be equally likely to come from two barcodes, and thus the error cannot readily be corrected. With a Hamming distance of 3, a single error can be detected and corrected. A Hamming distance greater than 3 makes it possible to detect multiple errors, but these are expected to be negligible since sequencer errors are rare, and a double error is doubly rare. For small barcodes, e.g. 3 nucleotides, only 4 different barcodes are possible that maintain Hamming distance 3. Thus, for the 3 nucleotide design (Table 2), Hamming distance of at least 2 was used so there could be 12 different barcodes.

TABLE 2*

| | | SEQ | | |
|---|---|---|---|---|
| | | ID | | FIG. |
| Name | IDT Ordering Sequence | NO. | Barcode | No. |
| L1-bc1 | /5Phos/ rACTT GAA AGATCGGAAGAGCACACG AT /iBiodT/ | 24 | TTC | 2D, 1000 ng |
| | AGA CGTGTGCTCTTCCGATCT TTC AAGrU /3Phos/ | | | 2F |
| | | | | 3ABCDE, G |
| L1-bc2 | /5Phos/ rACTT AGG AGATCGGAAGAGCACACG AT /iBiodT/ | 25 | CCT | 2D, 100 ng |
| | AGA CGTGTGCTCTTCCGATCT CCT AAGrU /3Phos/ | | | |
| L1-bc3 | /5Phos/ rACTT GTT AGATCGGAAGAGCACACG AT /iBiodT/ | 26 | AAC | 2D, 10 ng |
| | AGA CGTGTGCTCTTCCGATCT AAC AAGrU /3Phos/ | | | |
| L1-bc4 | /5Phos/ rACTT ACC AGATCGGAAGAGCACACG AT /iBiodT/ | 27 | GGT | |
| | AGA CGTGTGCTCTTCCGATCT GGT AAGrU /3Phos/ | | | |
| L1-bc5 | /5Phos/ rACTT CCA AGATCGGAAGAGCACACG AT /iBiodT/ | 28 | TGG | |
| | AGA CGTGTGCTCTTCCGATCT TGG AAGrU /3Phos/ | | | |
| L1-bc6 | /5Phos/ rACTT TTG AGATCGGAAGAGCACACG AT /iBiodT/ | 29 | CAA | |
| | AGA CGTGTGCTCTTCCGATCT CAA AAGrU /3Phos/ | | | |
| L1-bc7 | /5Phos/ rACTT TGT AGATCGGAAGAGCACACG AT /iBiodT/ | 30 | ACA | |
| | AGA CGTGTGCTCTTCCGATCT ACA AAGrU /3Phos/ | | | |
| L1-bc8 | /5Phos/ rACTT CAC AGATCGGAAGAGCACACG AT /iBiodT/ | 31 | GTG | 2C |
| | AGA CGTGTGCTCTTCCGATCT GTG AAGrU /3Phos/ | | | |
| L1-bc9 | /5Phos/ rACTT GCG AGATCGGAAGAGCACACG AT /iBiodT/ | 32 | CGC | |
| | AGA CGTGTGCTCTTCCGATCT CGC AAGrU /3Phos/ | | | |
| L1-bc10 | /5Phos/ rACTT CTA AGATCGGAAGAGCACACG AT /iBiodT/ | 33 | TAG | |
| | AGA CGTGTGCTCTTCCGATCT TAG AAGrU /3Phos/ | | | |
| L1-bc11 | /5Phos/ rACTT ATT AGATCGGAAGAGCACACG AT /iBiodT/ | 34 | AAT | |
| | AGA CGTGTGCTCTTCCGATCT AAT AAGrU /3Phos/ | | | |
| L1-bc12 | /5Phos/ rACTT GGC AGATCGGAAGAGCACACG AT /iBiodT/ | 35 | GCC | |
| | AGA CGTGTGCTCTTCCGATCT GCC AAGrU /3Phos/ | | | |
| Read1 L2 | /5Phos/NNN NNN GAT CGT CGG ACT GTA GAA /3ddC/ | 22 | | |
| oligo | | | | |

*Barcodes are 3 nucleotides, spaced by Hamming distance of at least 2.
The hairpin anneals to a read 1 primer.

TABLE 3**

| | | SEQ ID | | FIG. |
|---|---|---|---|---|
| Name | IDT Ordering Sequence | NO. | Barcode | No. |
| read1_bc1 | /5Phos/rACT GGAA GAT CGT CGG ACT GTA GAA CAT | 36 | TTCC | |
| | /iBiodT/ AG AGT TCT ACA GTC CGA CGA TC TTCC AG | | | |
| | rU/3Phos/ | | | |
| read1_bc2 | /5Phos/rACT CAGA GAT CGT CGG ACT GTA GAA CAT | 37 | TCTG | |
| | /iBiodT/ AG AGT TCT ACA GTC CGA CGA TC TCTG AG | | | |
| | rU/3Phos/ | | | |
| read1_bc3 | /5Phos/rACT ACCA GAT CGT CGG ACT GTA GAA CAT | 38 | TGGT | |
| | /iBiodT/ AG AGT TCT ACA GTC CGA CGA TC TGGT AG | | | |
| | rU/3Phos/ | | | |
| read1_bc4 | /5Phos/rACT TCAG GAT CGT CGG ACT GTA GAA CAT | 39 | CTGA | |
| | /iBiodT/ AG AGT TCT ACA GTC CGA CGA TC CTGA AG | | | |
| | rU/3Phos/ | | | |
| read1_bc5 | /5Phos/rACT ATGG GAT CGT CGG ACT GTA GAA CAT | 40 | CCAT | |
| | /iBiodT/ AG AGT TCT ACA GTC CGA CGA TC CCAT AG | | | |
| | rU/3Phos/ | | | |
| read1_bc6 | /5Phos/rACT GATG GAT CGT CGG ACT GTA GAA CAT | 41 | CATC | |
| | /iBiodT/ AG AGT TCT ACA GTC CGA CGA TC CATC AG | | | |
| | rU/3Phos/ | | | |
| read1_bc7 | /5Phos/rACT CTAC GAT CGT CGG ACT GTA GAA CAT | 42 | GTAG | |
| | /iBiodT/ AG AGT TCT ACA GTC CGA CGA TC GTAG AG | | | |
| | rU/3Phos/ | | | |
| read1_bc8 | /5Phos/rACT TACC GAT CGT CGG ACT GTA GAA CAT | 43 | GGTA | |
| | /iBiodT/ AG AGT TCT ACA GTC CGA CGA TC GGTA AG | | | |
| | rU/3Phos/ | | | |
| read1_bc9 | /5Phos/rACT AGTC GAT CGT CGG ACT GTA GAA CAT | 44 | GACT | 2E, S1 |
| | /iBiodT/ AG AGT TCT ACA GTC CGA CGA TC GACT AG | | | |
| | rU/3Phos/ | | | |
| read1_bc10 | /5Phos/rACT TGGT GAT CGT CGG ACT GTA GAA CAT | 45 | ACCA | 2E, S2 |
| | /iBiodT/ AG AGT TCT ACA GTC CGA CGA TC ACCA AG | | | |
| | rU/3Phos/ | | | |
| read1_bc11 | /5Phos/rACT GTCT GAT CGT CGG ACT GTA GAA CAT | 46 | AGAC | |
| | /iBiodT/ AG AGT TCT ACA GTC CGA CGA TC AGAC AG | | | |
| | rU/3Phos/ | | | |
| read1_bc12 | /5Phos/rACT CCTT GAT CGT CGG ACT GTA GAA CAT | 47 | AAGG | |
| | /iBiodT/ AG AGT TCT ACA GTC CGA CGA TC AAGG AG | | | |
| | rU/3Phos/ | | | |
| Read1 L2 | /5Phos/NNN NNN AGA TCG GAA GAG CAC ACG /3ddC/ | 23 | | |
| oligo | | | | |

**Barcodes are 4 nucleotides, spaced by Hamming distance of at least 3 (error correcting).
The hairpin anneals to a read 1 primer.

TABLE 4***

| | | SEQ ID | | FIG. |
|---|---|---|---|---|
| Name | IDT Ordering Sequence | NO. | Barcode | No. |
| read2_bc1 | /5Phos/rACT GGAA AGA TCG Gaa gag cac acg at | 48 | TTCC | 1 |
| | /iBiodT/ agaCGT GTG CTC TTC CGA TCT TTCC AG | | | |
| | rU/3Phos/ | | | |
| read2_bc2 | /5Phos/rACT CAGA AGA TCG Gaa gag cac acg at | 49 | TCTG | |
| | /iBiodT/ agaCGT GTG CTC TTC CGA TCT TCTG AG | | | |
| | rU/3Phos/ | | | |
| read2_bc3 | /5Phos/rACT ACCA AGA TCG Gaa gag cac acg at | 50 | TGGT | |
| | /iBiodT/ agaCGT GTG CTC TTC CGA TCT TGGT AG | | | |
| | rU/3Phos/ | | | |
| read2_bc4 | /5Phos/rACT TCAG AGA TCG Gaa gag cac acg at | 51 | CTGA | |
| | /iBiodT/ agaCGT GTG CTC TTC CGA TCT CTGA AG | | | |
| | rU/3Phos/ | | | |
| read2_bc5 | /5Phos/rACT ATGG AGA TCG Gaa gag cac acg at | 52 | CCAT | |
| | /iBiodT/ agaCGT GTG CTC TTC CGA TCT CCAT AG | | | |
| | rU/3Phos/ | | | |
| read2_bc6 | /5Phos/rACT GATG AGA TCG Gaa gag cac acg at | 53 | CATC | |
| | /iBiodT/ agaCGT GTG CTC TTC CGA TCT CATC AG | | | |
| | rU/3Phos/ | | | |
| read2_bc7 | /5Phos/rACT CTAC AGA TCG Gaa gag cac acg at | 54 | GTAG | |
| | /iBiodT/ agaCGT GTG CTC TTC CGA TCT GTAG AG | | | |
| | rU/3Phos/ | | | |
| read2_bc8 | /5Phos/rACT TACC AGA TCG Gaa gag cac acg at | 55 | GGTA | |
| | /iBiodT/ agaCGT GTG CTC TTC CGA TCT GGTA AG | | | |
| | rU/3Phos/ | | | |
| read2_bc9 | /5Phos/rACT AGTC AGA TCG Gaa gag cac acg at | 56 | GACT | |
| | /iBiodT/ agaCGT GTG CTC TTC CGA TCT GACT AG | | | |
| | rU/3Phos/ | | | |
| read2_bc10 | /5Phos/rACT TGGT AGA TCG Gaa gag cac acg at | 57 | ACCA | |
| | /iBiodT/ agaCGT GTG CTC TTC CGA TCT ACCA AG | | | |
| | rU/3Phos/ | | | |
| read2_bc11 | /5Phos/rACT GTCT AGA TCG Gaa gag cac acg at /iBiodT/ | 58 | AGAC | |
| | agaCGT GTG CTC TTC CGA TCT AGAC AG rU/3Phos/ | | | |
| read2_bc12 | /5Phos/rACT CCTT AGA TCG Gaa gag cac acg at /iBiodT/ | 59 | AAGG | |
| | agaCGT GTG CTC TTC CGA TCT AAGG AG rU/3Phos/ | | | |
| read2_bc13 | /5Phos/rACT GGAA AGA TCG Gaa gag cac acg at | 60 | TTCC | |
| | /iBiodT/ agaCGT GTG CTC TTC CGA TCT TTCC AG | | | |
| | rU/3Phos/ | | | |
| Read2 L2 | /5Phos/NNN NNN GAT CGT CGG ACT GTA GAA/3ddC/ | 22 | | |
| oligo | | | | |

***Barcodes are 4 nucleotides, spaced by Hamming distance of at least 3 (error correcting).
The hairpin anneals to a read 2 primer.

Table 5 provides oligonucleotide sequences used in the final PCR step of RNA-seq process. These oligonucleotides extend ˜5 bases past Illumina TruSeq™ Small RNA Index primers. The primers are used to make libraries compatible with Illumina sequencing platforms.

TABLE 5

| | Illumina | Index | | SEQ |
|---|---|---|---|---|
| Sample | small | (not | | ID |
| I.D. | rna number | Barcode) | Full sequence | NO. |
| PCR Illumina | Illumina | | AATGAACGGCGAC CACCGAGATCTAC | 61 |
| multiplex | multiplex | | ACGTTCAGAGTTC TACAGTCCGACGATC | |
| 1 | RPI1 | CGTGAT | CAAGCAGAAGACGGCATACGAGATCGTGATGT | 62 |
| | | | GACTGGAGTTCAGACGTGTGCTCTTCCGATCT | |
| 2 | RPI2 | ACATCG | CAAGCAGAAGACGGCATACGAGATACATCGGT | 63 |
| | | | GACTGGAGTTCAGACGTGTGCTCTTCCGATCT | |
| 3 | RPI3 | GCCTAA | CAAGCAGAAGACGGCATACGAGATGCCTAAGT | 64 |
| | | | GACTGGAGTTCAGACGTGTGCTCTTCCGATCT | |
| 4 | RPI4 | TGGTCA | CAAGCAGAAGACGGCATACGAGATTGGTCAGT | 65 |
| | | | GACTGGAGTTCAGACGTGTGCTCTTCCGATCT | |
| 5 | RPI5 | CACTGT | CAAGCAGAAGACGGCATACGAGATCACTGTGT | 66 |
| | | | GACTGGAGTTCAGACGTGTGCTCTTCCGATCT | |
| 6 | RPI6 | ATTGGC | CAAGCAGAAGACGGCATACGAGATATTGGCGT | 67 |
| | | | GACTGGAGTTCAGACGTGTGCTCTTCCGATCT | |
| 7 | RPI7 | GATCTG | CAAGCAGAAGACGGCATACGAGATGATCTGGT | 68 |
| | | | GACTGGAGTTCAGACGTGTGCTCTTCCGATCT | |
| 8 | RPI8 | TCAAGT | CAAGCAGAAGACGGCATACGAGATTCAAGTGT | 69 |
| | | | GACTGGAGTTCAGACGTGTGCTCTTCCGATCT | |
| 9 | RPI9 | CTGATC | CAAGCAGAAGACGGCATACGAGATCTGATCGT | 70 |
| | | | GACTGGAGTTCAGACGTGTGCTCTTCCGATCT | |
| 10 | RPI10 | AAGCTA | CAAGCAGAAGACGGCATACGAGATAAGCTAGT | 71 |
| | | | GACTGGAGTTCAGACGTGTGCTCTTCCGATCT | |
| 11 | RPI11 | GTAGCC | CAAGCAGAAGACGGCATACGAGATGTAGCCGT | 72 |
| | | | GACTGGAGTTCAGACGTGTGCTCTTCCGATCT | |
| 12 | RPI12 | TACAAG | CAAGCAGAAGACGGCATACGAGATTACAAGGT | 73 |
| | | | GACTGGAGTTCAGACGTGTGCTCTTCCGATCT | |
| 13 | RPI13 | TTGACT | CAAGCAGAAGACGGCATACGAGATTTGACTGT | 74 |
| | | | GACTGGAGTTCAGACGTGTGCTCTTCCGATCT | |
| 14 | RPI14 | GGAACT | CAAGCAGAAGACGGCATACGAGATGGAACTGT | 75 |
| | | | GACTGGAGTTCAGACGTGTGCTCTTCCGATCT | |
| 15 | RPI15 | TGACAT | CAAGCAGAAGACGGCATACGAGATTGACATGT | 76 |
| | | | GACTGGAGTTCAGACGTGTGCTCTTCCGATCT | |
| 16 | RPI16 | GGACGG | CAAGCAGAAGACGGCATACGAGATGGACGGGT | 77 |
| | | | GACTGGAGTTCAGACGTGTGCTCTTCCGATCT | |
| 17 | RPI17 | CTCTAC | CAAGCAGAAGACGGCATACGAGATCTCTACGT | 78 |
| | | | GACTGGAGTTCAGACGTGTGCTCTTCCGATCT | |
| 18 | RPI18 | GCGGAC | CAAGCAGAAGACGGCATACGAGATGCGGACGT | 79 |
| | | | GACTGGAGTTCAGACGTGTGCTCTTCCGATCT | |
| 19 | RPI19 | TTTCAC | CAAGCAGAAGACGGCATACGAGATTTTCACGT | 80 |
| | | | GACTGGAGTTCAGACGTGTGCTCTTCCGATCT | |
| 20 | RPI20 | GGCCAC | CAAGCAGAAGACGGCATACGAGATGGCCACGT | 81 |
| | | | GACTGGAGTTCAGACGTGTGCTCTTCCGATCT | |
| 21 | RPI21 | CGAAAC | CAAGCAGAAGACGGCATACGAGATCGAAACGT | 82 |
| | | | GACTGGAGTTCAGACGTGTGCTCTTCCGATCT | |
| 22 | RPI22 | CGTACG | CAAGCAGAAGACGGCATACGAGATCGTACGGT | 83 |
| | | | GACTGGAGTTCAGACGTGTGCTCTTCCGATCT | |
| 23 | RPI23 | CCACTC | CAAGCAGAAGACGGCATACGAGATCCACTCGT | 84 |
| | | | GACTGGAGTTCAGACGTGTGCTCTTCCGATCT | |
| 24 | RPI24 | GCTACC | CAAGCAGAAGACGGCATACGAGATGCTACCGT | 85 |
| | | | GACTGGAGTTCAGACGTGTGCTCTTCCGATCT | |

### 32P-Labeling

5′-end labeling: Radiolabeling reactions were performed by adding ³²P T4 PNK mix (final concentration of 1 U/μL T4 PNK, 30 mM imidazole-HCl buffer, 2.5 μM [15 μCi/μL] γ-³²P ATP, 1 mM ADP) to a solution of 5′-phosphorylated oligonucleotide (final concentration of 1.25 μM). The sample was incubated at 37° C. for 30 minutes; T4 PNK was then heat inactivated by incubating at 65° C. for 10 minutes.

dTTP incorporation: Reverse transcription was performed as described in the RNA-seq section, except that 5 μL of the sample in 1×SuperScript™ IV VILO mix were removed; to this, 1 μL of 10 μCi/μL α-³²P dTTP was added. After incubation, the sample was treated with 2 μL of 18 mg/ml proteinase K (Roche) before analysis by gel electrophoresis.

The following methods were used in one or more of the applications of RNA-seq libraries described in the Results.

E. coli Growth, Stress, RNA Extraction

E. coli MG1655 cells were grown in LB to a A600 of 0.4 before subjecting to the stress conditions. Mock treated cells, 25 mL, were left to grow for 10 min. Hydrogen peroxide stress was induced by adding H₂O₂ to 25 mL cells to a final concentration of 0.5% for 10 min. Glucose phosphate stress was induced by adding a-methyl glucoside-6-phosphate (αMG) to 25 mL of cells to a final concentration of 1 mM for 10 min. Iron depletion stress was induced by adding 2,2′-dipyridl (DIP) to 25 mL of cells to 250 μM final concentration for 10 min. Cells were harvested by centrifuging 25 mL culture for 1 min at 12 000 rcf and decanting media. Cells were resuspended in 0.5 mL ice cold lysis buffer (150 mM KCl, 2 mM EDTA, 20 mM HEPES pH 7.5) then flash frozen in liquid nitrogen. RNA was extracted by a hot acid-phenol protocol. Briefly, 0.5 mL of acid-buffer phenol (pH 4.5 citrate) was added to frozen samples. Samples were incubated in a heat block with shaking at 50° C. for 30 min. The aqueous phase was extracted for another round of phenol extraction and 2 rounds of chloroform extraction before ultimately precipitating with glycoblue, 300 mM sodium acetate, and 3 volumes of ethanol. Samples were incubated for 1 hour at −80° C., then centrifuged at maximum speed (20k RCF) for 45 min to pellet RNA. Pellets were washed twice with 70% ethanol, then resuspended in water.

### HEK Cell Culture and RNA Extraction

HEK293T cells were cultured with complete DMEM medium under standard conditions. Briefly, HEK293T cells were grown in Hyclone™ DMEM medium (GE Healthcare Life Sciences, SH30022.01) with 10% FBS and 1% Pen-Strep (Penicillin-Streptomycin) to 80% confluency and passaged. Cells were collected and total RNA was extracted using TRIzol™ (ThermoFisher, 15596026) by following the manufacturer's protocol when cells reached 80-90% confluency.

### MCF7 Growth and RNA Extraction

MCF7 cells were cultured in EMEM medium (ATCC, 30-2003) with 10% FBS (ThermoFisher, 10082147), 0.01 mg/ml bovine insulin (Sigma-Aldrich, 10516), and 10 nM β-estradiol (Sigma-Aldrich, E2758) to 80% confluency and passaged at ratios of 1:3. Total RNA was extracted using TRIzol™.

### Stool and Oral Sample Collection and RNA Extraction

Oral Cavity: Tongue dorsum scrapings were collected from 1 female and 3 male volunteers (two samples per volunteer) on two consecutive days [A & B sample]. Sample collection used BreathRx Gentle Tongue Scraper (Philips Sonicare) and was performed prior to eating, drinking or performing oral hygiene. Starting as far back as possible on the tongue, the scraper was passed forward over the entire surface three sequential times. The scrapings were combined with 500-μl RNAlater™ Stabilization solution (Invitrogen) and stored at −80° C. until extraction.

Gastrointestinal tract: Stool specimens were self-collected by 1 female and 1 male volunteer. Volunteers were provided with a commercial “toilet hat” stool specimen collection kit (Fisherbrand Commode Specimen Collection System; Thermo Fisher Scientific). Specimens were immediately transported to the laboratory (<1-hr) and thoroughly homogenized. 100-mg stool was transferred into a cryovial using a sterile spatula and 700-μl RNAlater Stabilization solution was then added. Specimens were stored at −80° C. until extraction.

Total RNA Extraction: RNA was later removed from tongue dorsum and stool samples by centrifugation at 17,200 rcf for 10 minutes at 4° C. Pelleted material was lysed in 400 μL of 0.3M NaOAc/HOAc, 10 mM EDTA, pH 4.8 with an equal volume of acetate-saturated phenol chloroform pH 4.8. After addition of 1.0 mm glass lysing beads (Bio-Spec Products, Bartlesville, OK) in a 1:1 ratio (bead:sample weight), samples were placed in a reciprocating bead beater (Mini-Beadbeater-16, Bio-Spec Products) for two 1-min intervals on maximum intensity. Samples were centrifuged at 17,200 rcf for 15 minutes at 4° C. before re-extraction and isopropanol precipitation of total RNA. Pellets were washed with 75% ethanol before resuspension in an acid-buffered elution buffer (10 mM NaOAc, 1 mM EDTA, pH 4.8).

### AlkB and AlkB D135S Purification

These protocols were adapted from the previously described protocols for DM-tRNA-seq (Zheng et al., Nature Methods 12, 835, 2015). Briefly, NEB T7 Expression cells were grown in LB media at 37° C. in the presence of 50 μM kanamycin to an A600 of 0.6-0.8. Once the cells reached the desired density, IPTG and iron sulfate were added to final concentrations of 1 mM and 5 μM, respectively. After induction, the cells were incubated overnight at 30° C. Cells were collected, pelleted and then resuspended in lysis buffer (10 mM Tris, pH 7.4, 5% glycerol, 2 mM CaCl₂, 10 mM MgCl₂, 10 mM 2-mercaptoethanol) plus 300 mM NaCl. The cells were lysed by sonication and then centrifuged at 17,400×g for 20 min. The soluble proteins were first purified using a Ni-NTA superflow cartridge (Qiagen) with buffers A (lysis buffer plus 1 M NaCl for washing) and B (lysis buffer plus 1 M NaCl and 500 mM imidazole for elution) and then further purified by ion-exchange (Mono S GL, GE Healthcare) with buffers A (lysis buffer plus 100 mM NaCl for column loading) and B (lysis buffer plus 1.5 M NaCl for elution).

### Poly(A)-Selection

Poly(A)-selection for HEK mRNA sequencing was done with NEBNext® Poly(A) mRNA Magnetic Isoloation Module (Catalog #: E7490S) according to manufacturer's instructions.

### AlkB Treatment Conditions

Demethylase buffer conditions were modified from those published (Li et al., Nat Struct Mol Biol 25, 1047, doi:10.1038/s41594-018-0142-5, 2018). Three stock solutions were made fresh immediately before reaction: L-ascorbic acid 200 mM, 2-ketogluterate 3 mM, and ammonium iron sulfate 5 mM. The final reaction buffer contained 2 mM L-ascorbic acid, 1 mM 2-ketogluterate, 0.3 mM ammonium iron sulfate, 100 mM KCl, 50 mM MES pH 6, 50 ng/μL BSA, 4 μM wild-type AlkB, and 4 μM AlkB-D135S. 50 μL of the reaction mixture was added to 5-20 μL of decanted streptavidin bead slurry after ligation, immobilization, and washing. Reaction continued for 30 min at 37° C. Following the reaction, beads were washed once with high salt wash buffer (20 mM TrisHCl pH 7.4, 1 M NaCl, 0.1% Tween20) and once with low salt wash buffer (20 mM TrisHCl pH 7.4, 100 mM NaCl).

### CMC Treatment/Library Construction

MCF7 total RNA sequencing libraries were constructed as follows. Small RNA (<200 nt) was first removed from 1 μg MCF7 total RNA using spin columns (Zymo RNA Clean & Concentrator™-5, R1016) and the large RNA (>200 nt) was eluted with 18 μl sterile H₂O in a microcentrifuge tube. The RNA was transferred to PCR tubes and 2 μl Magnesium RNA fragmentation buffer (NEB, E6150S) were added to each tube and the tubes were incubated at 94° C. in a thermocycler for 5 minutes to fragment the RNA to ˜200 nt. 2 μl RNA fragmentation stop solution were then added to each tube. The samples were diluted to 50 μl with H2O and Zymo spin columns were used to purify the fragmented RNA; the RNA were eluted in 16 μl sterile H₂O in a microcentrifuge tube. For 3′-end repair of the RNA fragments, 2 μl 10× T4 PNK buffer and 2 μl T4 PNK at 10U/μl (ThermoFisher, EK0032) were added and the mixture incubated at 37° C. for 30 minutes. The fragmented, end-repaired RNA was used to build sequencing libraries using the RNA-seq protocol described above with the following modifications. The fragmented RNA was ligated to bar-coded hairpin oligonucleotides and bound to streptavidin beads. The samples were then pooled, mixed and split into two parts for ±CMC (N-cyclohexyl-N′-(2-morpholinoethyl)carbodiimide) treatment (+CMC:−CMC=1.5:1 ratio). 12 μl sterile H2O and 24 μl TEU buffer (50 mM Tris-HCl (pH 8.3), 4 mM EDTA, 7 M urea) were first added to each tube, then 4 μl freshly prepared 1M CMC in TEU buffer was added to +CMC samples and 4 μl sterile H₂O were added −CMC samples. The samples were incubated at 30° C. for 16 hours at 1400 rpm (revolutions per minute) on an Eppendorf ThermoMixer. The samples were washed twice with high salt buffer and once with low salt buffer. The samples were then resuspended with 40μl of 50 mM sodium carbonate and 2 mM EDTA (pH 10.4) buffer and incubated at 37° C. for 6 hours at 1400 rpm. The beads were washed twice with high salt buffer and once with low salt buffer and then proceeded to the RNA-seq steps such as phosphatase treatment and reverse transcription.

tRNA Microarrays

The tRNA microarrays consist of four processes starting from purified tRNA or total RNA without the need of cDNA synthesis: (i) deacylation, (ii) selective fluorophore labeling of tRNA using oligonucleotide ligation with T4 DNA ligase to the 3′-CCA of all tRNA, (iii) hybridization and (iv) data analysis. The reproducibility of the E. coli and human tRNA microarray method and validation of the results have been extensively described previously (Dittmar et al., EMBO Rep 6, 151, 2005; Pavon-Eternod et al., Nucleic Acids Res 37, 7268, doi:gkp787[pii]10.1093/nar/gkp787, 2009).

### Read Processing and Mapping

Libraries were sequenced on Illumina Hi-Seq or NEXT-seq platform. Paired-end reads were combined with bbmerge from the JGI BBtools toolset. Reads were merged such that the sample barcode was oriented at the start of a read: for libraries constructed with the read-2 barcodes, the order of read1 and read2 were flipped for bbmerge inputs. Next, merged reads, one file for each index, were split by barcode using fastX toolkit barcode splitter. Custom python scripts (available on GitHub) were used to remove the barcode sequence (first 7 nt) and to collapse reads using the UMI, then remove the UMI (last 6 bases). Next reads were mapped using bowtie2 with the “local” parameter. Human samples were mapped either to a curated list of mature tRNAs predicted from tRNA-scan SE with a score greater than 40, augmented with “CCA” endings added where needed, or to a genome combining ensemble HG19 orfs, ncRNAs, and curated tRNA. E. coli samples were mapped to either a curated list of non-redundant tRNAs from tRNA-scan SE with score >40 and CCA added where needed, or a combine E. coli genome including ensemble ORFs, and ensemble ncRNAs which included tRNA genes. Bowtie2 output sam files were converted to bam files, then sorted using samtools. Next IGV was used to collapse reads into 1 nt window. IGV output.wig files were reformatted using custom python scripts (available GitHub). The bowtie2 output Sam files were also used with eXpress from pachter lab to sum all reads that mapped to each gene. Data was visualized with custom R scripts (GitHub).

The read counts and mapping rates are provided in Table 6.

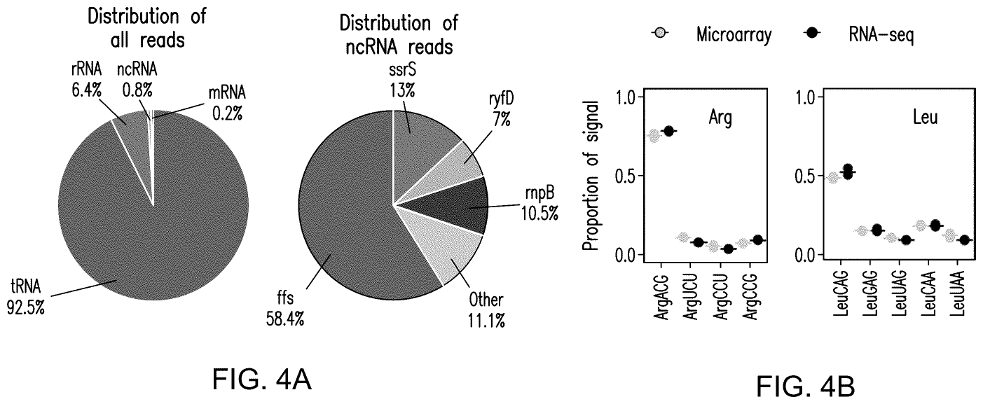

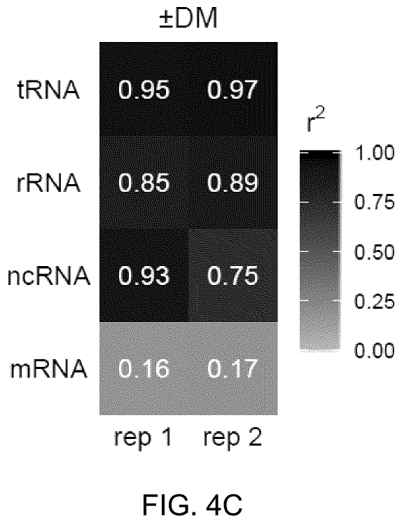

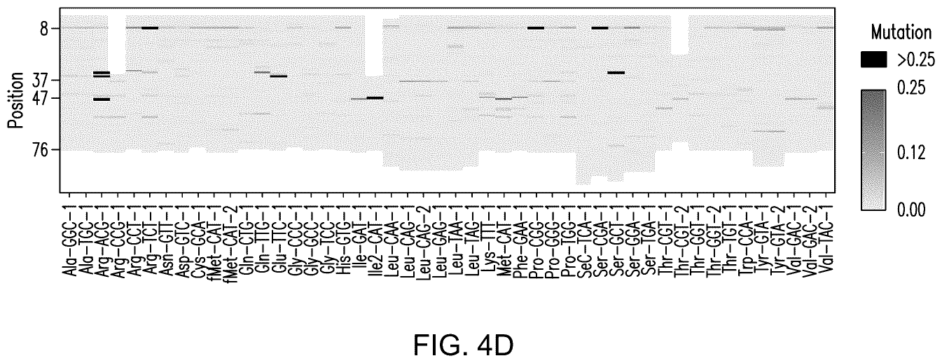

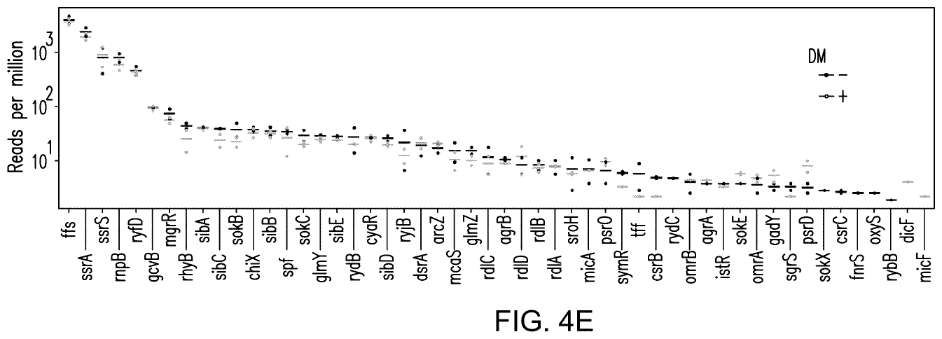

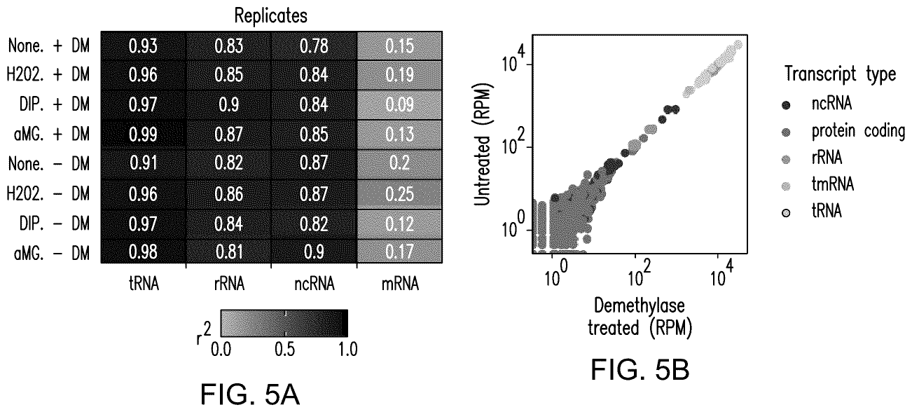

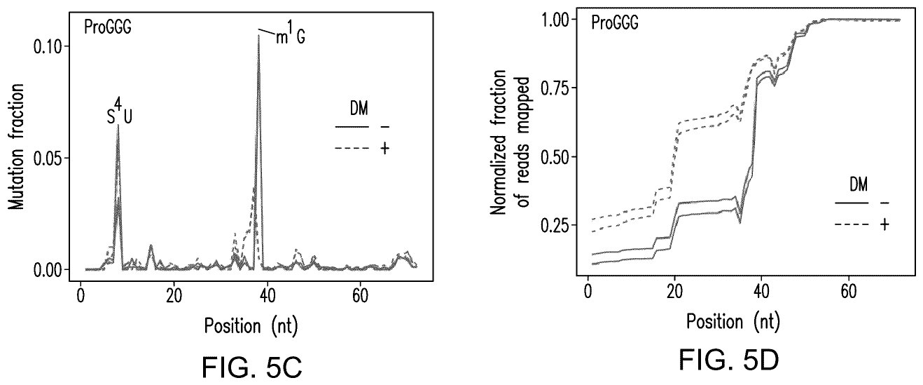

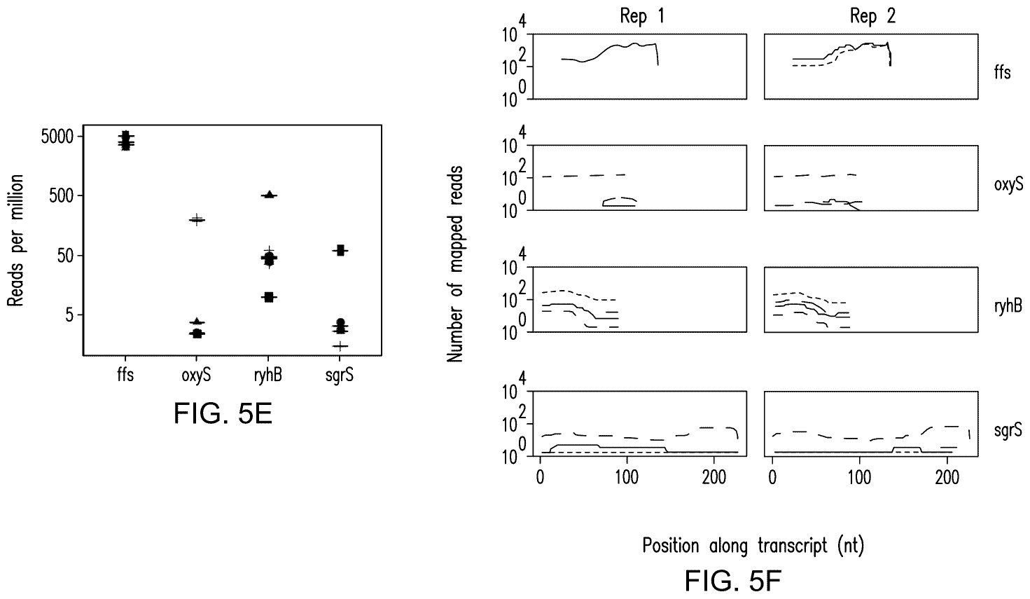

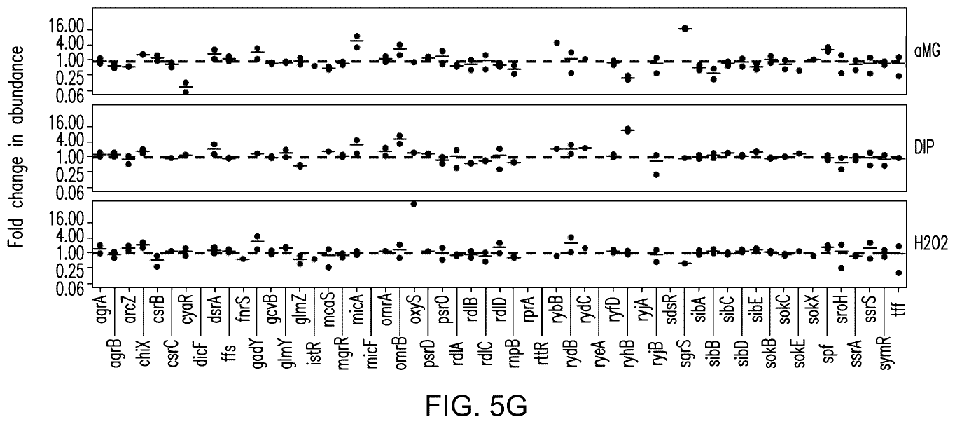

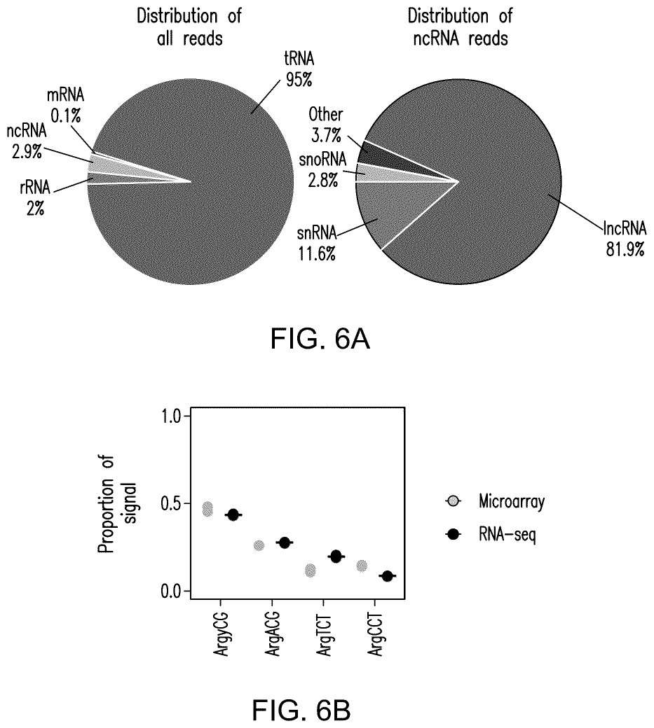

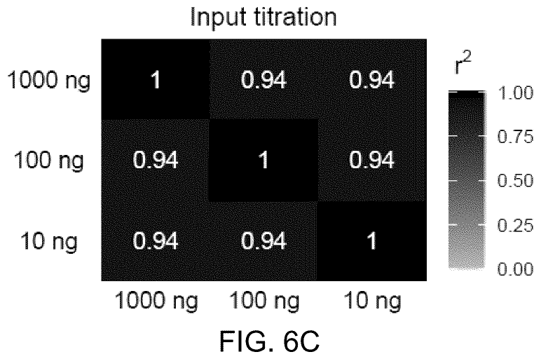

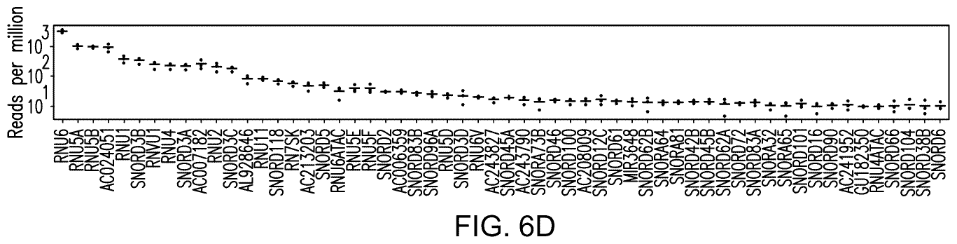

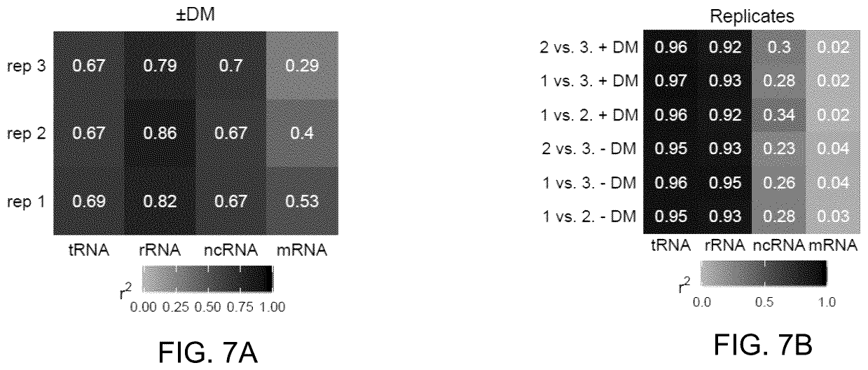

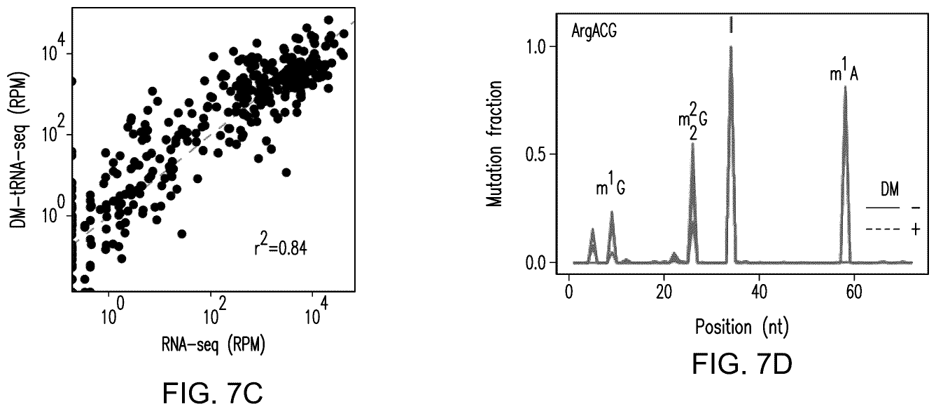

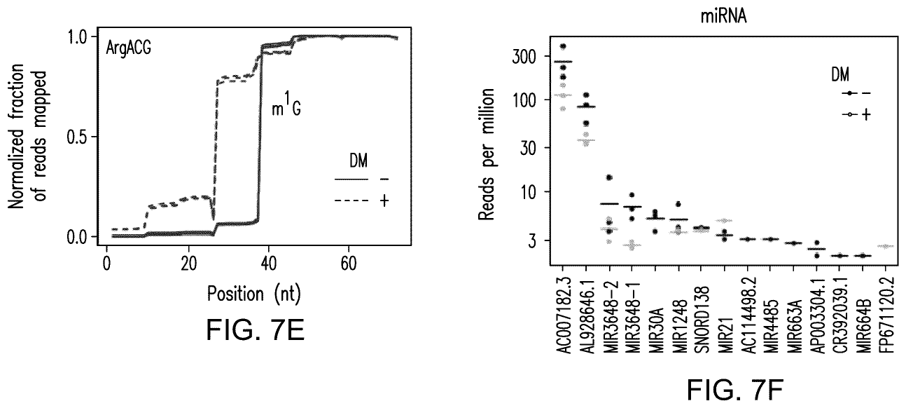

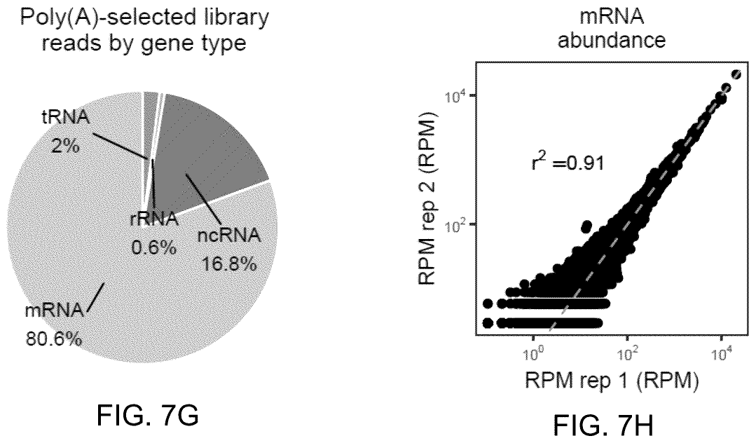

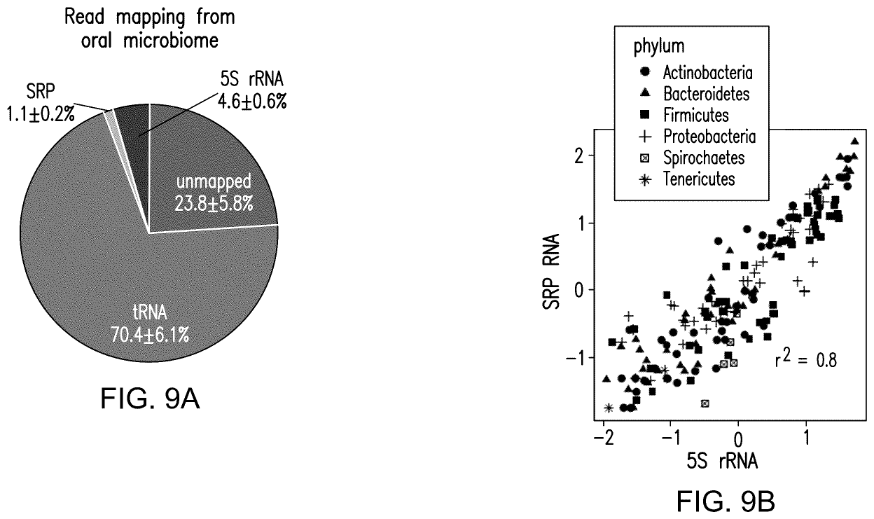

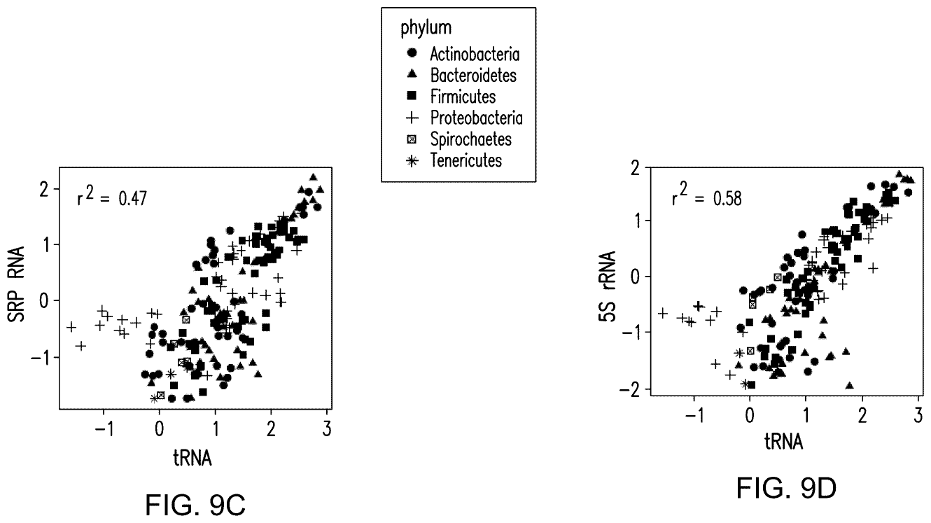

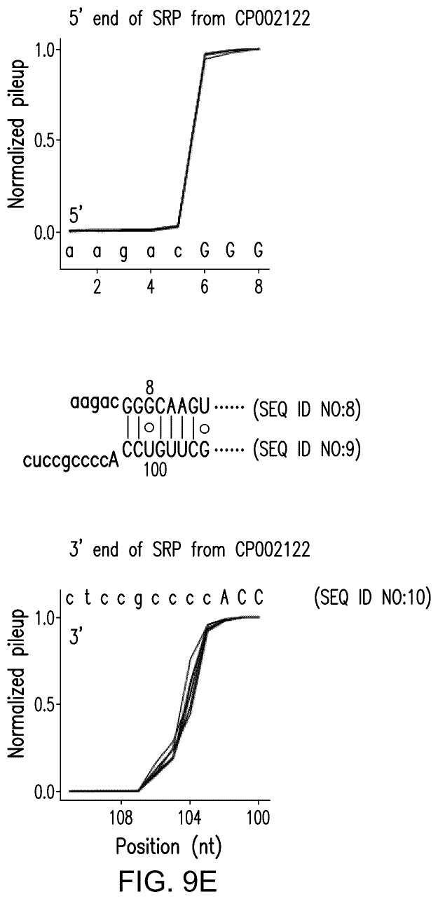

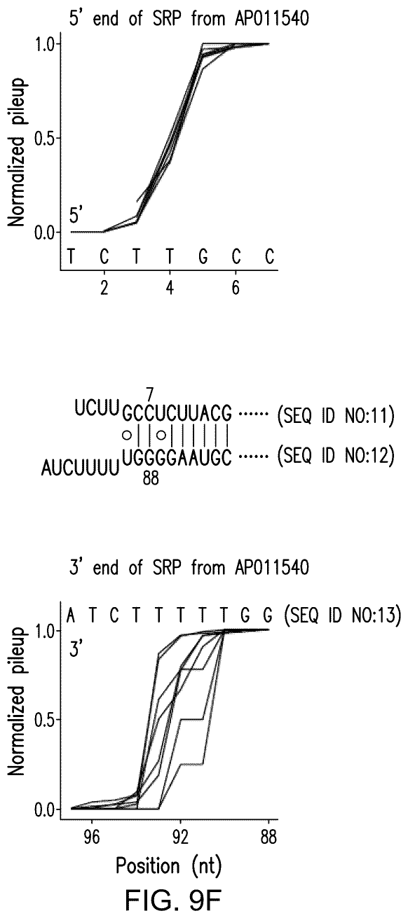

TABLE 6

| | Sample # | | After | | |
|---|---|---|---|---|---|
| | in one Hi- | | collapse | Mapped | Mapping |
| Experiment | seq lane | Sample | reads | reads | rate |
| E. coli | | | | | |
| FIG. 4 | 48 | Rep1, −DM, none | 1337283 | 1078299 | 0.806 |
| | | Rep1, +DM, none | 1126882 | 917433 | 0.814 |
| | | Rep2, −DM, none | 987286 | 805777 | 0.816 |
| | | Rep2, +DM, none | 606555 | 496693 | 0.819 |
| FIG. 5 | 48 | Rep1, −DM, DIP | 898022 | 769002 | 0.856 |
| | | Rep1, +DM, DIP | 784402 | 673001 | 0.858 |
| | | Rep2, −DM, DIP | 645502 | 530346 | 0.822 |
| | 48 | Rep2, +DM, DIP | 404885 | 334063 | 0.825 |
| | | Rep1, −DM, aMG | 1442022 | 1245524 | 0.864 |
| | | Rep1, +DM, aMG | 1249915 | 1079328 | 0.864 |
| | | Rep2, −DM, aMG | 1487242 | 1310364 | 0.881 |
| | | Rep2, +DM, aMG | 957734 | 844986 | 0.882 |
| | 48 | Rep1, −DM, H202 | 1673105 | 1355992 | 0.810 |
| | | Rep1, +DM, H2O2 | 1352966 | 1106744 | 0.818 |
| | | Rep2, −DM, H202 | 1653616 | 1332915 | 0.806 |
| | | Rep2, +DM, H202 | 999083 | 810908 | 0.812 |
| HEK293T | | | | | |
| FIG. 6 | 34 | Rep1, −DM | 1397160 | 1088669 | 0.779 |
| | | Rep1, +DM | 1121900 | 1035782 | 0.923 |
| | | Rep2, −DM | 1288756 | 982480 | 0.762 |
| | | Rep2, −DM | 907752 | 823781 | 0.907 |
| | | Rep3, −DM | 1437164 | 1071859 | 0.746 |
| | | Rep3, −DM | 866332 | 782064 | 0.903 |
| MCF7 | | | | | |
| FIG. 7 | 24 | Rep1, −CMC | 10406306 | 9056121 | 0.870 |
| | | Rep1, +CMC | 4933523 | 4065387 | 0.824 |
| | | Rep2, −CMC | 17037349 | 14825014 | 0.870 |
| | | Rep2, +CMC | 3460452 | 2853392 | 0.825 |
| Stool, tongue | | | | | |
| FIG. 9 | 12 | Stool 1, −DM | 67668773 | 65544672 | 0.969 |
| | | Stool 1, +DM | 48839683 | 47278949 | 0.968 |
| | | Stool 2, −DM | 5970288 | 5431084 | 0.910 |
| | | Stool 2, +DM | 4387984 | 4026445 | 0.918 |
| | 48 | Tongue 1, −DM | 3111780 | 3047099 | 0.979 |
| | | Tongue 1, +DM | 2892388 | 2816590 | 0.974 |
| | | Tongue 2, −DM | 2241862 | 2195573 | 0.979 |
| | | Tongue 2, −DM | 2034383 | 1989134 | 0.978 |

Read Processing from CMC Reaction

Raw 100 bp paired-end sequencing reads were obtained from Illumina Hi-Seq platform. Read1 reads were separated by barcodes with the barcodes sequence on paired read2 reads using custom python scripts. Read2 reads were separated by barcodes using fastx_barcode_splitter (fastx_toolkit, http://hannonlab.cshl.edu/fastx_toolkit/). For read1 reads, the random 6 nucleotide unique molecular identifier (UMI) sequence at the start of the reads and the barcoded adaptor sequence at the end of the reads were removed using Trimmomatic using single-end mode with a 15 nt cutoff. For read2 reads, the 7 nt barcode sequence at the start of the reads and the UMI and adaptor sequence at the end of the reads were removed by Trimmomatic using paired-end mode with a 15 nt cutoff. The reads were then mapped to human rRNA transcripts using bowtie2. The output sam files were converted to bam files and then sorted and indexed using samtools. Command-line version of “igvtools count” (IGV, http://software.broadinstitute.org/software/igv/download) were used to count nucleotide composition, insertions, and deletions at single base resolution. “Bedtools genomecov” (bedtools, https://bedtools.readthedocs.io/en/latest/) was used to count the start and end of all reads at each position. All the output files and reference sequence were combined into a single file for each sample, the mutation rate and the stop rate were computed by custom python scripts. The output files were analyzed to identify target pseudouridine sites.

Microbiome tRNA Analysis

These were modified from previously published pipeline with significant modifications. Raw paired-end sequence reads of 75 or 100 nucleotides were processed by Illumina-utils (available at https://github.com/merenlab/illumina-utils). Inserts contained a 7 nucleotide sample barcode and a random 6 nucleotide unique molecular identifier (UMI). Given that tRNA molecules range in length from 74-96 nucleotides, forward and reverse 100 nucleotide reads fully covered some tRNA sequences and partially overlapped for others. The Illumina-utils ‘iu-merge-pairs’ command was upgraded to merge both fully and partially overlapping reads, while trimming overhanging adapter sequences in the case of more than full overlap (the flag, ‘--marker-gene-stringent’, enables consideration of full as well as partial overlap). Erroneous base calls were minimized, which was important for the analysis of modification-induced mutations, by retaining reads that matched with zero mismatches in the overlapping region (option ‘-max-num-mismatches 0’).

Tools were developed in the Anvi'o multi-omics platform to identify tRNA sequences from reads (available at https://github.com/merenlab/anvio), including a Snakemake workflow to automate many of the following steps. The command ‘anvi-gen-tRNAseq -database’ runs a dynamic programming algorithm (module ‘trnaidentifier’) to profile tRNA features in reads and thereby select mature and fragmentary tRNA along with other related species such as pre-tRNA. All reads in the method started from 3′-CCA, so a set of minimum criteria for tRNA selection were defined that included acceptor nucleotides and the correct length to conserved nucleotides in the T arm, of which 5 of 7 were to be found. The algorithm continues searching for features, including the anticodon loop, toward the 5′-end of the read, with a full-length read containing a base-paired acceptor stem and all features in between. The algorithm searches each possible sequence upon encountering features that may be of variable length, such as the variable (V) loop, and returns the feature profile with the minimum sum of: “unconserved” nucleotides at canonically conserved positions and base pair mismatches in stems.

tRNA sequences were taxonomically annotated by using the GAST tool to search a set of reference tRNA sequences that tRNAscan-SE (v1.3.1) identified from 4,235 gold-standard bacterial genomes (non-endosymbiont genomes with an assembly level of “chromosome”) stored in the Ensembl Genomes 2016 database.

Specific nucleotide positions were selected from tRNA sequences for modification analysis. Positions were identified relative to features profiled by Anvi'o. For example, canonical position 22, a site of m1A modification in many tRNA species, is identified as being 5 nucleotides from the 5′-nucleotide of the anticodon stem, canonical position 27. Anvi'o workflow analyzed the distribution of nucleotides at positions of interest in each taxon, grouping tRNA species by anticodon. tRNA species were selected that were represented by at least 50 reads in both demethylated and untreated sample splits. Mutations likely to be caused by modifications were separated from other sources of nucleotide variants, such as related tRNA sequences with a single nucleotide polymorphism, by only considering tRNA species with 3 different nucleotides in at least 5% of reads from the untreated split. A significantly reduced mutation signature in the demethylated split confirmed the putative modification (χ² p-value<0.001, from the χ² test comparing the observed numbers of the 4 nucleotides in the demethylated experiment to the expected numbers of the 4 nucleotides given the distribution from the untreated experiment).

### Results

### RNA-Seq Process

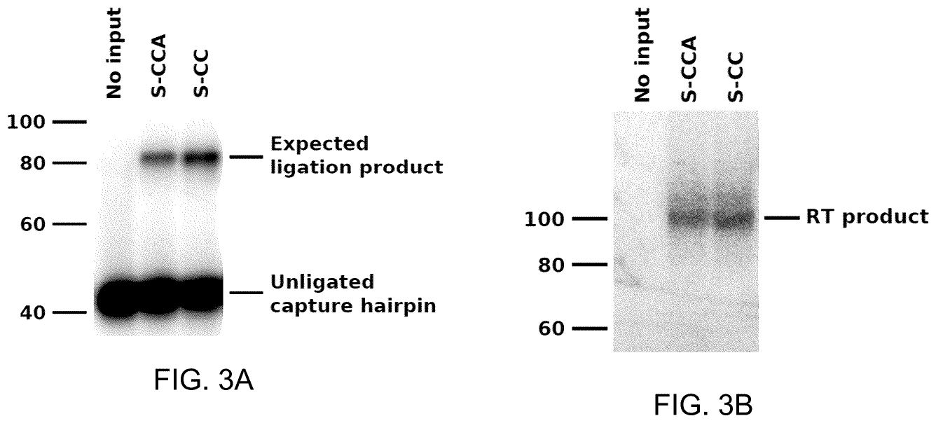

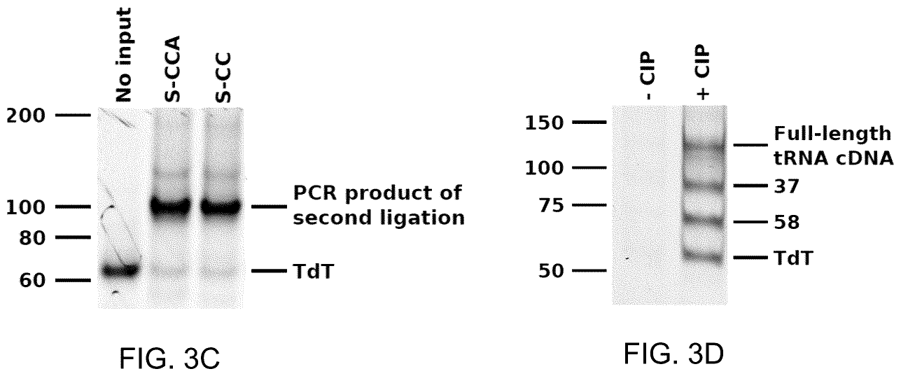

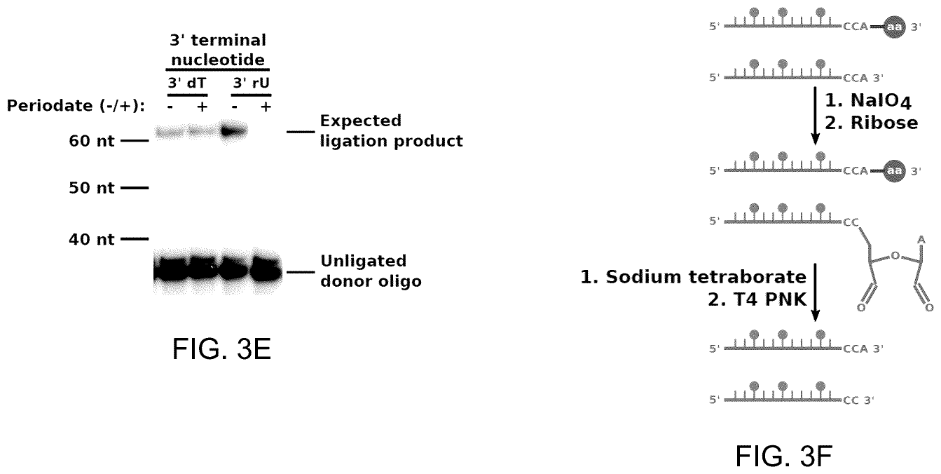

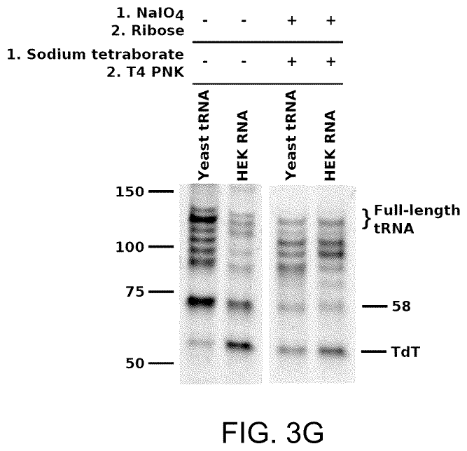

FIGS. 2C-F and FIGS. 3A-G display the results of experiments performed to explore various aspects of the RNA-seq platform and of using the platform in RNA-seq library preparation. The input material in the experiments was total RNA from HEK293T cells, unless otherwise noted. The figures show images of electrophoresis gels analyzing reaction products. DNA size markers are indicated on the left. Major RT (reverse transcriptase) stops caused by the m1A58 and m1G37 modifications in human tRNAs are indicated on the right. TdT corresponds to the product derived from the aberrant terminal transferase activity of the RT.

The ligation with T4 RNA ligase I was compatible with the duplex structure of the CHO and showed no bias between RNA substrates with 3′-A or 3′-C ends (FIG. 3A), a property needed for the charged tRNA measurements discussed later.

After streptavidin bead binding of all the CHO, some with the input RNA ligated and others without, the sample can be split in two for optional enzyme treatment. In this case, one sample was exposed to an AlkB demethylase mixture to remove Watson-Crick face methylations in tRNA, and the other was left untreated as a control. The on-bead enzyme reaction was highly efficient, as shown by the removal and reduction of the m1A58 and m1G37 bands, respectively, in the tRNA sample (FIG. 2C).

Reverse transcription using the thermostable Superscript™ IV RT was not inhibited by immobilization on beads (FIG. 3B). After on-bead second adaptor ligation to the cDNA product, PCR was directly performed on-bead to generate off-bead products ready for sequencing (FIG. 3C). 3′-phosphate was removed on-bead using alkaline phosphatase to allow for subsequent reverse transcription from the 3′-OH (FIG. 3D). It was confirmed that periodate treatment prevented ligation to a CHO with 3′-terminal ribose but had no effect on the same oligonucleotide with a 3′-terminal deoxyribose, as shown in FIG. 3E.

All but the first barcode ligation reaction was performed on-bead. This facilitated the removal of excess reagents in every step with simple washes, significantly reduced sample loss during each step, and allowed for construction of RNA-seq libraries with as little as 10 ng of total RNA input (FIG. 2D).

The RNA-seq protocol also generated high quality RNA-seq libraries from total nucleic acids isolated from complex samples such as human stool (FIG. 2E) or human tongue (FIG. 2F). The considerable amounts of DNA present in these samples did not interfere with library construction, with or without added DNase treatment (FIG. 2E). Stool sample S1* was first treated with DNase I before the first ligation step, whereas S1 and S2 used the same samples without DNase treatment. Samples (+) periodate were periodate oxidized before the first ligation step, which prevented RNA-CHO ligation. The m1A58 band seen in the HEK293T library is nearly absent in the stool sample libraries, suggesting that human tRNAs are present in low amounts in the nicrobiome sequencing libraries.

As a design goal for tRNA charging studies, the oxidation and beta-elimination protocol was modified to enable sequential addition of these reagents in a single tube so that no reaction intermediates were precipitated or purified, depicted schematically in FIG. 3F. The final mixture was used directly in CHO ligation. FIG. 3G shows the final PCR products without (−,−) and with (+,+) the treatments shown in FIG. 3F.

Total E. coli RNA

The use of RNA-seq in studying total RNA from E. coli is shown here. Though initially designed with tRNAs in mind, the RNA-seq system in principle is capable of detecting other types of RNA. Libraries were built from total E. coli RNA. Final PCR products were size selected for cDNA inserts between 15-150 nucleotides for sequencing.

FIGS. 4 and 5 depict the results of several analyses from the sequencing of total E. coli RNA.

In FIG. 4A, the RNA-seq results were mapped to the E. coli genome. As expected, the majority of reads align to mature tRNA (92%), while the remaining reads aligned to rRNA, non-coding RNA (ncRNA), and mRNA. A small fraction of the reads map to non-coding RNAs. In the absence of stress, ncRNA reads were mostly partitioned among a few abundant RNA species, including the well-characterized ffs (SRP RNA), ssrS (6S RNA), and rnpB (RNase P RNA) (FIG. 4A). The proportion of reads roughly reflects the molar ratios of cellular RNA transcripts in each category, in which tRNA makes up 80-90% on a molar basis. In the absence of stress, ncRNA reads were mostly partitioned among a few abundant RNA species including the well-characterized ffs (SRP RNA), ssrS (6S RNA), and rnpB (RNase P RNA) (FIG. 4A). Given the large differences in transcript coverage, the abundance from biological replicates correlated well for tRNA (r2>0.95), rRNA (r2>0.85), and ncRNA (r2>0.75), but was low for mRNA (FIG. 4C). The quantitative nature of tRNA abundance measurements obtained by sequencing was validated by comparison to those obtained by microarray hybridization for the isoacceptor families of tRNAArg and tRNALeu (FIG. 4B; light-colored dots on left in each pair are microarray data, dark-colored dots on right in each pair are RNA-seq data).

Because tRNAs are highly modified in bacteria, the RNA samples were treated on-bead with an AlkB-demethylase mixture, which efficiently removes Watson-Crick face methylations of N1-methyladenosine (m1A), N1-methylguanosine (m1G), and N3-methylcytosine (m3C) in human tRNAs. m1A and m3C are absent in E. coli tRNA, so the demethylase treatment may only affect the seven E. coli tRNAs containing m1G 20. As expected, the abundance of tRNA correlated well at the global level, with and without treatment with a mixture of AlkB demethylases (r2>0.95), whereas the correlation for RNA classes rRNA, ncRNA and mRNA fell within the same range as for biological replicates (FIG. 4C). The low correlation for mRNA is due to their low read counts.

FIG. 4D depicts a heatmap of mutation fractions along individual tRNAs, and reveals a small number of sites with high mutation fractions. It is well established that RNA modifications at the Watson-Crick face frequently leave mutation signatures in cDNA because of RT read-through. RT can also stop at the modified nucleotide. Depending on the chemical nature of the modification and the specific RT used in sequencing, mutation and stop fractions at individual modification sites can vary widely. Most of the sites correspond to known modifications such as inosine (I), 2-thiocytosine (s2C), 4-thiouridine (s4U), N1-methylguanosine (m1G) and 3-(3-amino-3-carboxypropyl)uridine (acp3U). The m1G modifications sensitive to demethylase treatment were analyzed first (FIGS. 5C-D). Superscript™ IV RT read through m1G some of the time, but stopped at high frequency. Demethylase treatment removed the methylation, resulting in a substantial decrease in mutations and stop fractions. Compared to the TGI RT used in analysis of a RNA-seq library prepared by a conventional method (Zheng et al., Nature Methods 12, 835, 2015), SuperScript™ IV RT has a lower mutation rate, but a higher stop rate at m¹G.

Other E. coli tRNA modifications at the Watson-Crick face include 4-thiouridine (s4U) at position 8, 2-thiocytosine (s2C) at position 32, and bulky modifications such as lysidine at anticodon wobble position 34, 2-methylthio-N6-isopentenyladenosine (ms2i6A) at position 37, and as 3-(3-amino-3-carboxypropyl)uridine (acp3U) at position 47. These modifications had very large differences in mutation and stop fractions (FIGS. 4D and 5C). The bulky 34 and 37 modifications had the highest stop fractions. Both acp3U and m1G had comparable mutation fractions accompanied with substantial stops. Both s4U and s2C were detected by mutation without any stops (FIGS. 4D and 5C). For s4U8, the mutation fraction was highly variable among different tRNAs, which may reflect the differences in their modification fractions under this biological condition. Much higher mutation levels were observed for s2C32 modifications, which may reflect both their high modification levels and an idiosyncratic property of Superscript™ IV RT. s2C32 was not detected in E. coli tRNA in a previous study using a different RT from thermophilic group II intron 3.

Approximately 50 non-coding RNAs were observed in E. coli that varied by ˜2,000-fold in expression levels (FIG. 4E). In the absence of stress, these were dominated by several conserved bacterial RNA species such as SRP RNA (ffs), tmRNA (ssrA), and RNase P RNA (rnpB), but the vast majority were expressed at much lower levels, consistent with their expected role in stress response. FIG. 4E depicts the abundance of non-coding RNA transcripts at rpm>1. The data shows that demethylase treatment has only a minor effect.

This and following experiments demonstrate the simultaneous analysis of tRNA and small non-coding RNA. Because of the extremely high levels of tRNA, small RNA sequencing has commonly been performed by size-selecting RNA away from tRNA. By starting with total RNA, this approach incorporates all RNA types in a single library according to their approximate molar ratios.

E. coli Stress Response

The application of RNA-seq in studying a biological response by subjecting E. coli to three acute stress conditions is shown here. Addition of: H₂O₂ corresponds to oxidative stress, 2,2′-dipyridyl (DIP) to iron starvation, and α-methyl glucoside-6-phosphate (aMG) to glucose starvation.

FIGS. 5A-G depict the results of sequencing total RNA from E. coli subjected to the three acute stress conditions.

FIG. 5A shows correlation of RNA transcript abundance among biological replicates of total RNA from E. coli grown in LB, with and without three acute stress conditions for 10 minutes. The abundance correlation agrees well for tRNA, rRNA and ncRNA, but not for mRNA, due to the very low coverage of mRNA. FIG. 5B shows the relationship between transcript abundance of samples treated with demethylase and untreated. FIG. 5C shows mutation rate along tRNA^{Pro}(GGG) from libraries with and without demethylase treatment. The untreated sample shows mutation peaks at known m¹G37 and s⁴U8 modifications. The m¹G37 mutation is prevented by demethylase treatment, while the s⁴U8 mutation is unaffected. FIG. 5D shows read density along tRNA^{Pro} (GGG), with and without demethylase treatment, which demonstrates a strong stop at m¹G37 which is mostly eliminated by demethylase treatment. The results shown in FIGS. 5A-D for E. coli grown in stress conditions mirror those discussed earlier for unstressed E. coli (FIGS. 4A-D).

A major bacterial response to stress is the upregulation of specific non-coding RNAs. The stress-responsive sequences analyzed in FIG. 5E were: OxyS (+), responsive to oxidative stress; rhyB (triangle), responsive to iron starvation; sgrS (squares), responsive o glucose starvation; and ffs (SRP; circles), unresponsive control sequence. FIG. 5F depicts coverage density of the 3 stress-responsive small non-coding RNAs and control RNA SRP (ffs) during stresses and unstressed as control (none). For each stress a dramatic increase in the expression of specific RNAs was detected: ˜75-fold increase in oxyS for oxidative stress, ˜10-fold increase in ryhB for iron starvation, and ˜60-fold increase in sgrS for glucose starvation (FIGS. 5E-G). The level of a control sequence, ffs (SRP RNA), remained unchanged under all conditions (FIGS. 5E-F).

FIG. 5G depicts fold change in abundance of all detected small non-coding RNAs from libraries without demethylase treatment; only a small number of transcripts responded to individual stresses, consistent with the literature.

Changes in tRNA abundance, charging and modification were also studied under the same stress conditions: oxidative stress, iron starvation and glucose starvation. Changes in tRNA abundance under these acute stress conditions (10 min) were within 1.3-fold. When analyzing the mutation rate along individual tRNAs, widespread hyper-modification at position 8 was observed after aMG and DIP stresses only, while hypo-modification at position 32 resulted only from DIP stress.

Changes in most tRNA charging levels were also small and within the bulk range, with the exception of tRNAs for serine and glycine. In all three stress conditions tRNA^{Ser} charging level increased by up to 1.8-fold; all 4 tRNA^{Ser} isoacceptors followed the same trend. This result is consistent with the known low levels of tRNA^{Ser} charging under culture condition used before stress. In the other direction, tRNA^{Gly} isoacceptor charging level changes were below the bulk range by up to 1.7-fold.

These results suggest that acute E. coli stress response through tRNA occurs more rapidly through tRNA charging than changes in tRNA abundance. However, it is possible that large changes in tRNA abundance could take over as the stress persists.

How stress affects tRNA modifications was also investigated. Among the four modifications that could be analyzed with high confidence using comparative mutation fractions between each stress and unstressed control, it was found that m1G37 levels changed little under stress, but acp3U47 levels increased in all three stress conditions. In contrast, substantial changes in both s2C32 and s4U8 levels depended on the stress condition. S2C32 level dropped only under iron starvation. S4U8 level increased under iron starvation and glucose starvation, but not under oxidative stress. The precise role and mechanism of these changes are not immediately clear.

### HEK293T RNA

The application of RNA-seq in studying total human RNA from HEK293T RNA is shown here.

FIGS. 6 and 7 depict the results of several analyses from the sequencing of total human RNA.

RNA-seq libraries were built with human total RNA (FIG. 6A). As expected, most reads were from tRNA (95%), with the remaining were from ncRNA (2.9%), rRNA (2%) and mRNA (0.1%). The ncRNA reads included lncRNAs, snRNAs, snoRNAs, and others, with most being lncRNAs and snRNAs. The quantitative nature of tRNA abundance obtained bydemethylase-treated libraries was validated by comparison to those obtained by microarray hybridization for the isoacceptor family of tRNA^{Arg} (FIG. 6B; light-colored dots on left in each pair are microarray data, dark-colored dots on right in each pair are RNA-seq data).

Human tRNAs have multiple Watson-Crick face methylations in many tRNA species. These include m1A at position 58, m1G at position 37, m3C at position 32, 2,2-dimethylguanosine (m22G) at position 26, and m1G at position 9. Therefore, demethylase treatment can have a large effect on tRNA abundance measurement. Indeed, comparing sequencing results with and without demethylase treatment, the overall abundance of tRNAs correlated only moderately (FIG. 7A, r2 ˜0.68), despite the excellent correlation of biological replicates with and without demethylase treatment (FIG. 7B, r2>0.95). This discrepancy could in part be attributed to the increased ambiguity of read assignments to specific human tRNAs and/or to the over-representation of hypomodified tRNAs in the untreated samples.

Comparing the sequencing result of RNA-seq with a previously published result from DM-tRNA-seq (Zheng et al., Nature Methods 12, 835, 2015) showed a good correlation (FIG. 7C). The main differences between the inventive RNA-seq and the previous DM-tRNA-seq were use of different RT enzymes, steps involved in library construction, and the input of total RNA in RNA-seq versus gel purified-tRNA in DM-tRNA-seq.

The robustness of the RNA-seq method was tested by building libraries starting with 10, 100 and 1000 ng of total RNA (FIGS. 2D and 6C). FIG. 6C depicts correlation of tRNA abundance results from libraries starting with 1 μg, 100 ng, or 10 ng total RNA. Even at 10 ng total RNA input, tRNA abundance was well correlated between these libraries with r2 ˜0.94.

The extensive tRNA modification landscape was readily apparent by analysis of mutation fractions along individual tRNAs, which revealed many sites with high mutation fractions. Most of the mutation sites corresponded to known modifications, such as N1-methyladenosie (m1A) at position 58, N1-methylguanosine (m1G) at position 37, N3-methylcytosine (m3C) at position 32, N2,2-dimethylguanosine (m22G) at position 26, and m1G/m1A at position 9. With the exception of m1G37, essentially all methylations at the Watson-Crick face produced high mutation fractions across a tRNA sequence (FIG. 7D).

Mutation fractions were analyzed in tRNAs after demethylase treatment. As expected, all major changes were from demethylase-sensitive modifications sites such as m1A, m1G and m3C (see FIG. 7E). Demethylase treatment abolished or diminished the mutation and stops associated with these modifications in both nuclear-encoded and mitochondrial-encoded tRNAs, while the inosine (I) modification at the wobble anticodon position of many tRNAs was not affected.

In addition to tRNA, many small non-coding RNAs were also identified (FIG. 6D). Their abundance varied by ˜2,000-fold. FIG. 6D depicts the abundance of small non-coding RNA transcripts at rpm>10. As expected, most of these are spliceosomal RNAs and snoRNAs, plus a few abundant micro-RNAs, shown in FIG. 7F. tRNA fragments were not analyzed here and were excluded in this category.

Although RNA-seq was initially designed to study small RNAs, it can in principle be used to study mRNA. Sequencing libraries were also prepared using as input poly(A)-selected and then fragmented RNA. In this case, the majority of reads indeed mapped to mRNA and poly-adenylated ncRNA (97%), with only a small fraction mapping to tRNA (2%) and rRNA (0.6%) (FIG. 7G). Replicates correlated well for mRNA (r2=0.91), supporting the usefulness of the RNA-seq method for transcriptome sequencing (FIG. 7H).

### Pseudouridine (Ψ) Site Mapping by Chemical Treatment

The robustness of the on-bead protocol for applications involving harsh chemical treatment of RNA is shown here.

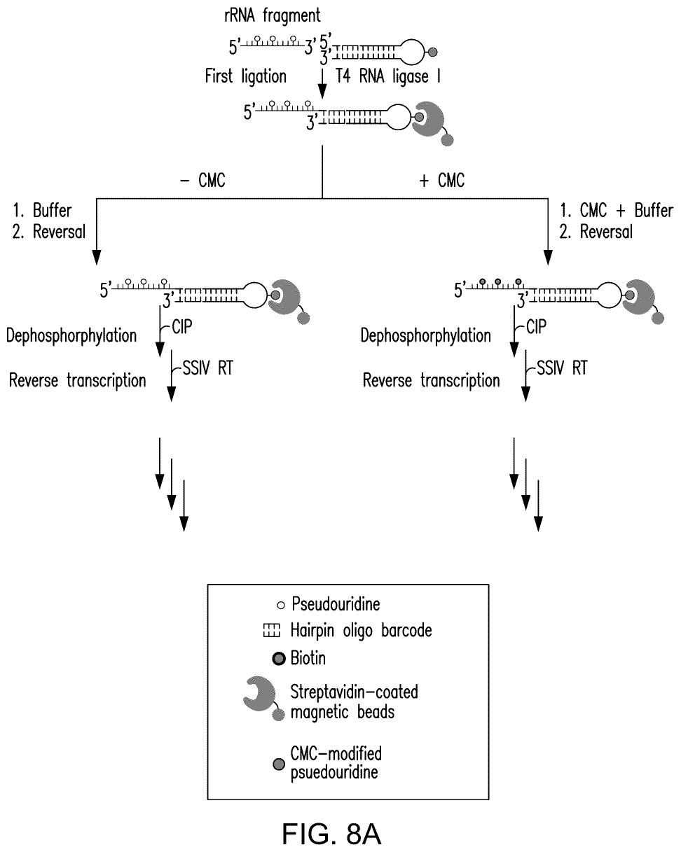

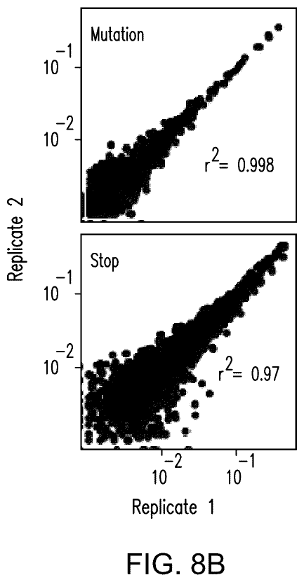

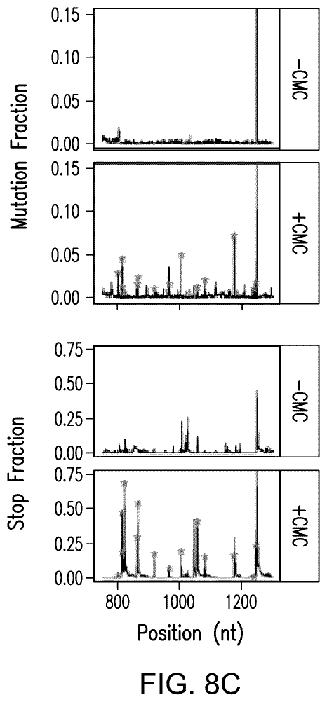

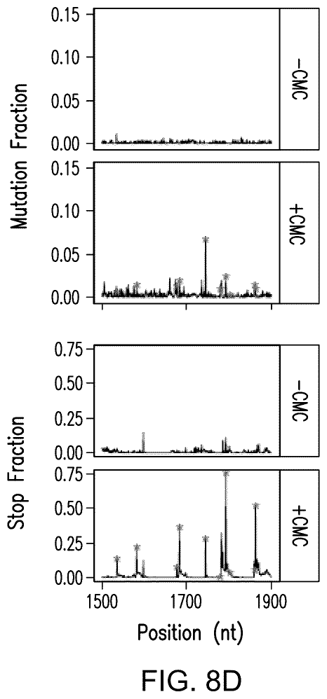

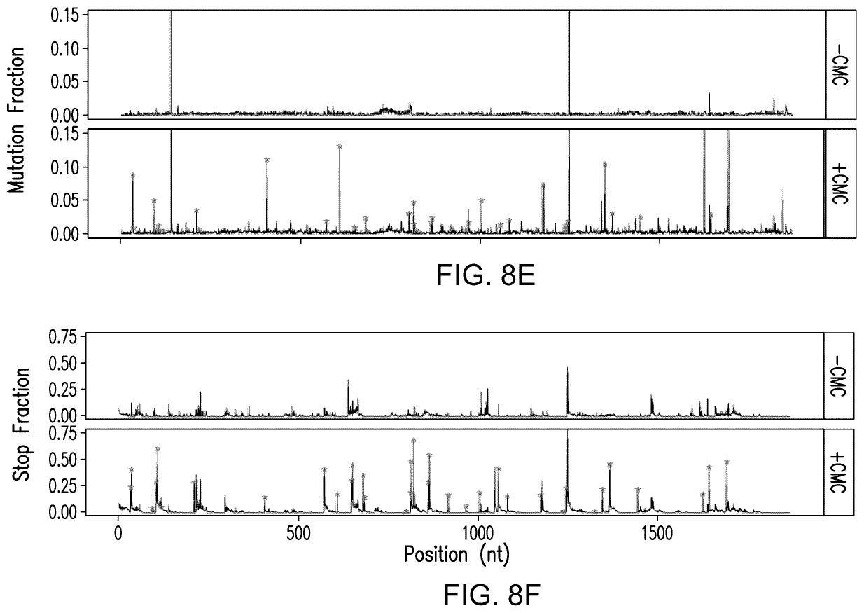

FIG. 8 depicts the use of RNA-seq to explore Ψ sites in human rRNA.

Chemical treatment of RNA has many applications, such as RNA structural mapping or identification of RNA modifications. A well-established method to identify Ψ sites is the reaction using N-cyclohexyl-N′-β-(4-methylmorpholinium) ethylcarbodiimide (CMC). Ψs are detected by increased RT stops and/or mutations at the Ψ site found when comparing a CMC-treated sample with an untreated control

Human rRNA has ˜100 known Ψ sites. In order to map them, total RNA was chemically fragmented, 3′-end repaired, then ligated to the hairpin oligonucleotide. The on-bead demethylation step was replaced with the CMC reactions in building the sequencing libraries (FIG. 8A). Each rRNA position was assigned a stop and a mutation fraction, and good correlation was observed between the biological replicates (r2>0.95) (FIG. 8B). The regions in the 18S (FIG. 8C) and 28S (FIG. 8D) rRNA known to be rich in Ψ sites were examined, as well as full-length 18S rRNA (FIGS. 8E-F). All known Ψ sites are indicated by asterisks in FIGS. 8C-F. Strong signals were identified in the stop and/or mutation fractions in the CMC-treated samples at known Ψ sites, validating the usefulness of the approach.

This example shows that the streptavidin beads can withstand harsh chemical treatments such as the CMC reaction, which involves two steps carried out at pH 8-10 and hours of incubation at 30-37° C.

Microbiome tRNA Sequencing

The usefulness of the RNA-seq approach in studying complex samples, such as microbiomes, is shown here.

FIGS. 9-12 depict the use of RNA-seq to explore the microbiomes in human stool and tongue.

Most microbiome characterization techniques sequence DNA, which can determine community membership but not microbial activity. Previous work developed a microbiome tRNA-seq approach (Schwartz et al., Nat Commun 9, 5353, doi:10.1038/s41467-018-07675-z, 2018) that measured tRNA expression and tRNA modification in the mouse cecum. However, the previous method has many limitations, including requiring a large amount of input material and gel purification of tRNAs before, and cDNA products during, library construction.

The E. coli and human cell lines used in the previous studies were from defined cultures, in which the amount of input sample was practically unlimited and the data complexity was low, as each sample could be aligned to a single reference genome. In contrast, samples from human stool and tongue are far more complex. Having demonstrated that RNA-seq libraries from these samples were of good quality (see FIGS. 2E-F), the RNA-seq libraries were used in sequencing and the data analyzed for tRNA abundance and modification using the de novo tRNA-seq pipeline developed previously. For stool and tongue samples, >95% of all tRNA-compatible reads were assigned to bacteria, indicating that the procedure produced high value results for microbiome characterization.

FIG. 9A shows the assignment of reads to different major RNA classes from a human tongue scraping. FIG. 9B shows the correlation of SRP RNA and 5S rRNA from various bacterial taxonomic classes. Values are computed as the Z-score of log10 abundance. FIG. 9C shows the correlation of SRP RNA abundance and the sum of all identified tRNAs for bacterial taxonomic classes, as in B. FIG. 9D shows the correlation of 5S rRNA and the sum of all identified tRNAs for bacterial taxonomic classes as in B. FIG. 9E shows reads mapping to SRP of Prevotella melaninogenica; reads map to the annotated 5′-end (top) of the gene (capitol letters), whereas the 3′-end of the transcript (bottom) 1-3 bases beyond the gene annotation into the genomic sequence (lowercase letters); extended 3′-end is consistent with the SRP structural context (middle). FIG. 9F shows reads mapping to SRP of Rothia mucilaginosa; reads map to 2-5 bases downstream of the annotated 5′-end (top) of the gene, while the 3′-end (bottom) shows heterogenaity between individuals with the 3′-end varying by 4-8 nt short of the annotated end.

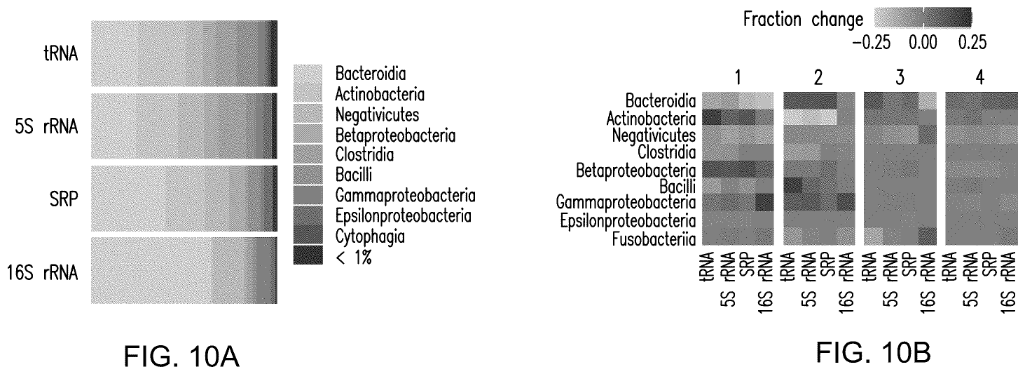

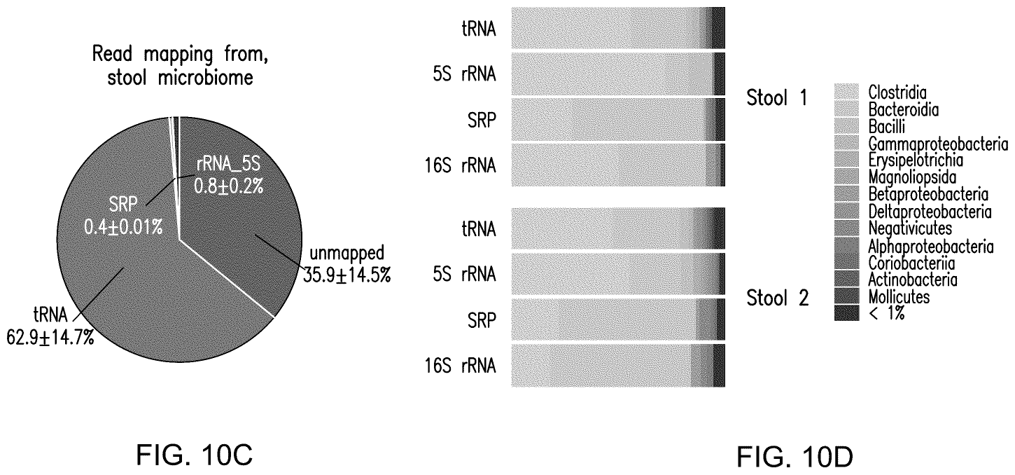

FIG. 10A shows the taxonomic composition of microbes from a human tongue scraping calculated using either tRNA, 5S rRNA, SRP RNA, or measured by 16S amplicon gene sequencing; Actinobacteria are known to evade detection by 16S amplicon sequencing explaining variation between RNA and 16S DNA sequencing techniques. FIG. 10B shows the fold change in tongue microbe abundance between 2 sequential days for 4 different individuals, as measured by tRNA, 5S rRNA, SRP RNA, and 16S amplicon sequencing. FIG. 10C shows read assignment to different major RNA classes from human stool. FIG. 10D shows the taxonomic composition of microbes from two human stool samples calculated using either tRNA, 5S rRNA, SRP RNA, or measured by 16S amplicon gene sequencing.

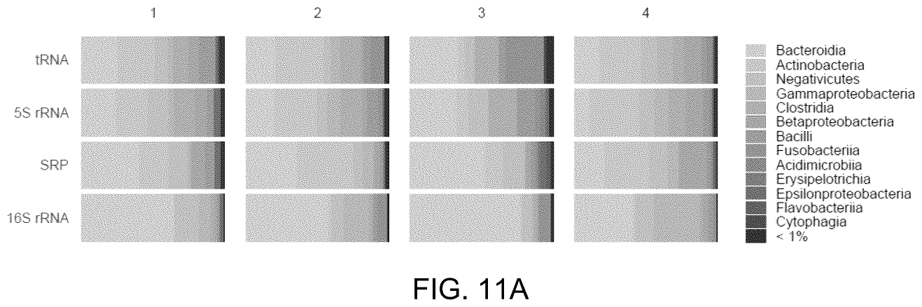

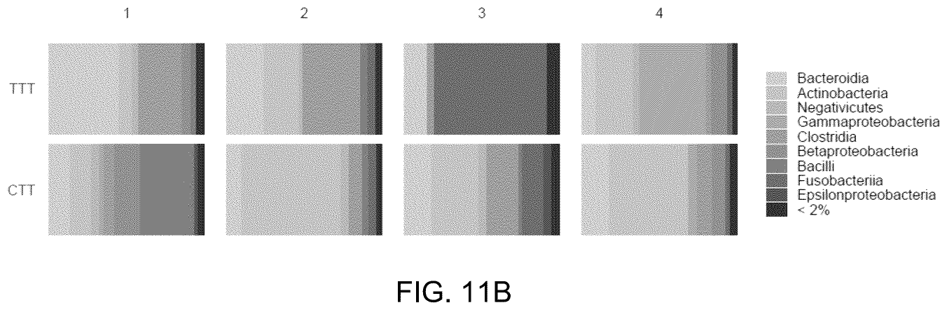

FIG. 11A shows the taxonomic composition of microbes from 4 different human tongue scrapings calculated using either tRNA, 5S rRNA, SRP RNA, or measured by 16S amplicon gene sequencing. FIG. 11B shows the taxonomic composition of microbes from a human tongue scraping calculated using either tRNAs bearing anticodon either “TTT” or “CTT”.

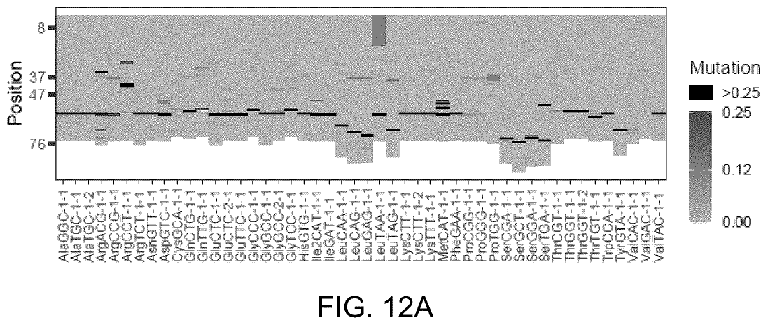

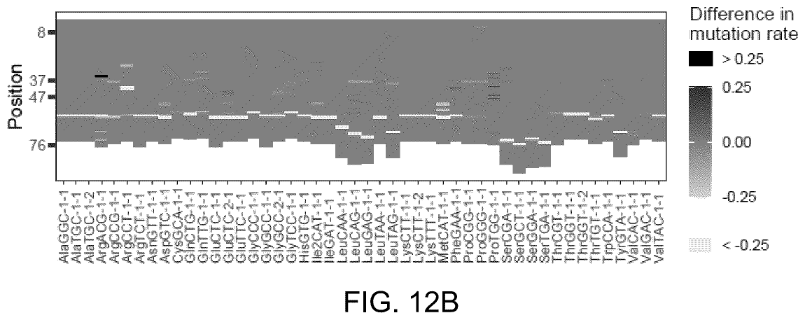

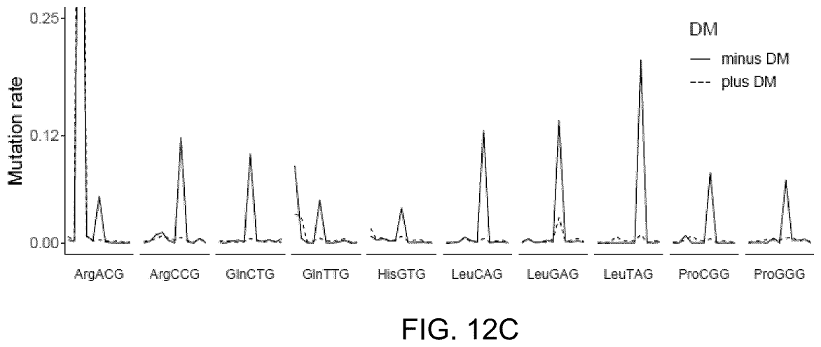

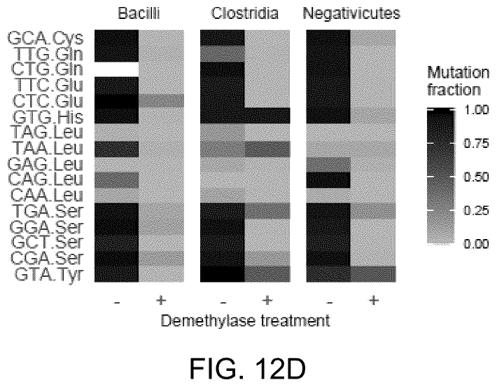

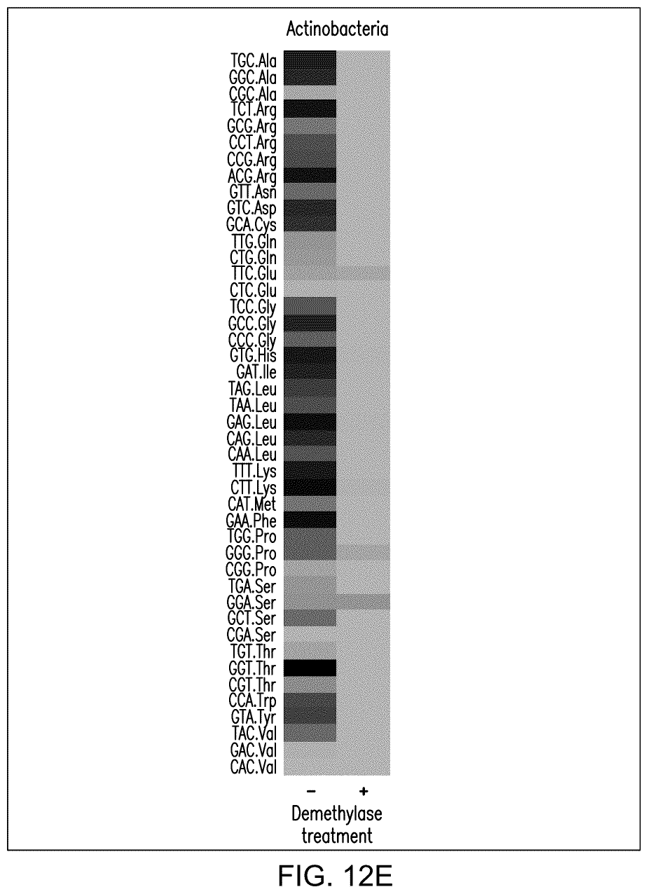

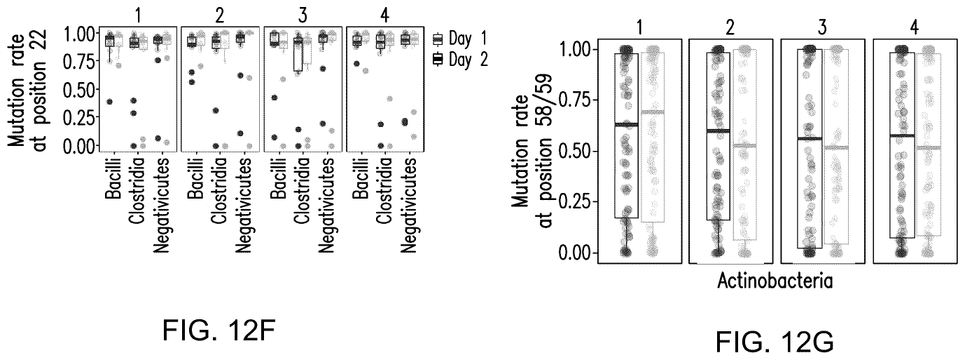

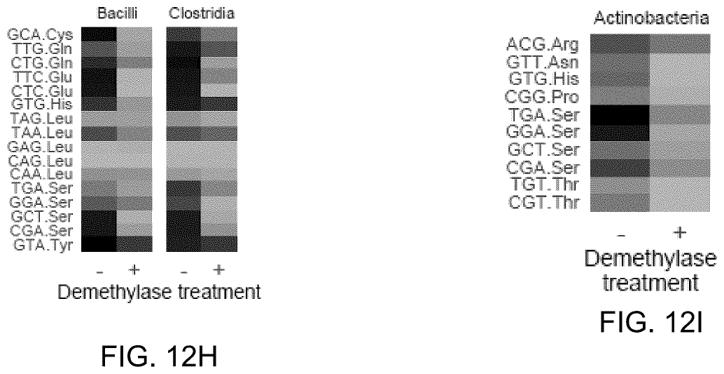

tRNA modifications were also analyzed. FIG. 12A shows a heat map of mutation rates along individual tRNAs of bacteria from the genus Rothia from human tongue scraping. FIG. 12B shows a heat map as in A, but identifies mutations that are sensitive to demethylase treatment and identifies abundant m1A58 modification in this genus. FIG. 12C shows the mutation rate at position 37 and surrounding bases of select tRNAs from genus Rothia and identifies m1G37 as a demethylase sensitive modification. FIG. 12D shows mutation rate at position 22 from select tRNAs in several bacterial taxons from human tongue with and without demethylase treatment, which identifies modification m1A22. FIG. 12E identifies m1A58 in Actinobacteria from human tongue as in D. FIG. 12F shows the mutation rate at position 22 for select bacterial classes without demethylase treatment from 4 human tongue scrapings on 2 sequential days. FIG. 12G shows the mutation rate at position 58 for Actinbacteria without demethylase treatment from 4 human tongue scrapings on 2. FIG. 12H identifies m1A22 in select bacteria classes as in D, from human stool. FIG. 12I identifies m1A58 in Actinobacteria as in E, from human stool.

RNA-seq improves the application of microbiome tRNA-seq in several ways, including the ability to handle many samples at once, a very substantial reduction in the amount of input sample, elimination of all size selection steps, and on-bead demethylase reaction.

### EXAMPLE 2

### SARS-CoV-2

The following Example demonstrates use of the RNA-seq method of RNA library preparation, which is generally described in Example 1 and uses a hairpin oligonucleotide as described herein, for the development of a potential SARS-CoV-2 biomarker, in accordance with aspects of the invention.

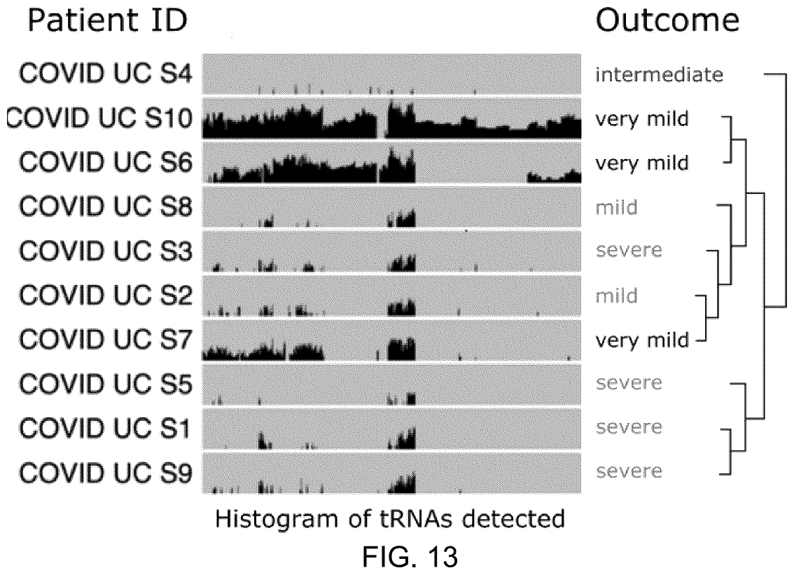

FIG. 13 depicts a histogram of tRNAs detected in samples obtained from the noses of SARS-CoV-2-infected individuals.

Ten nasal swab samples were obtained from individuals previously diagnosed with SARS-CoV-2. The RNA-seq method was applied to detect human and microbial tRNAs in the samples. Blinded clustering analysis was performed based on the tRNAs detected, and compared with patient outcomes, determined by length of hospital stay. The main clusters correspond well to severe symptoms (>15 days in hospital) and mild/very mild symptoms (<3 days).

Nasopharyngeal swabs from SARS-CoV-2 patients and healthy individuals as controls were sequenced to determine the quality of sequencing data that could be obtained from nasopharyngeal swabs used for COVID19 testing. These samples are low-biomass and contain only small amounts of RNA that are often undetectable by standard UV absorbance measurements. Although low sample biomass is not an issue for qPCR-based diagnostics, it represents an obstacle for most RNA-sequencing technology.

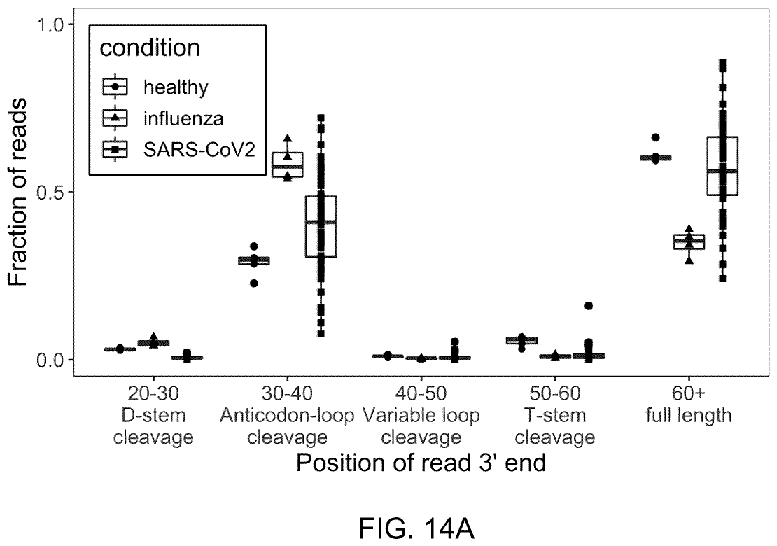

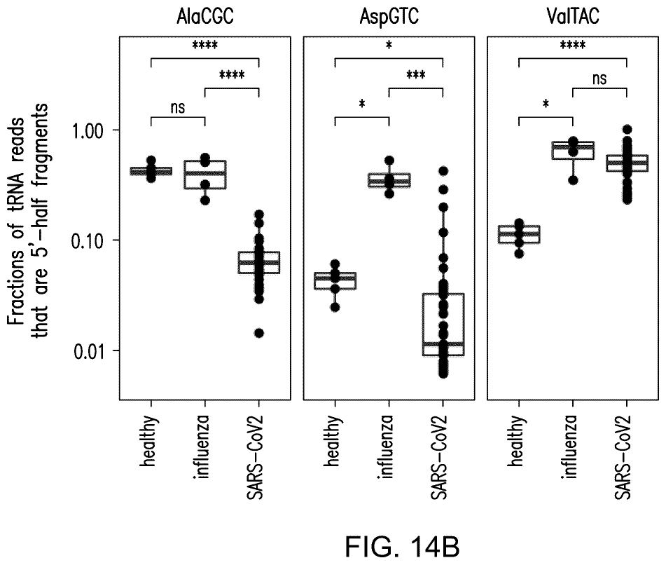

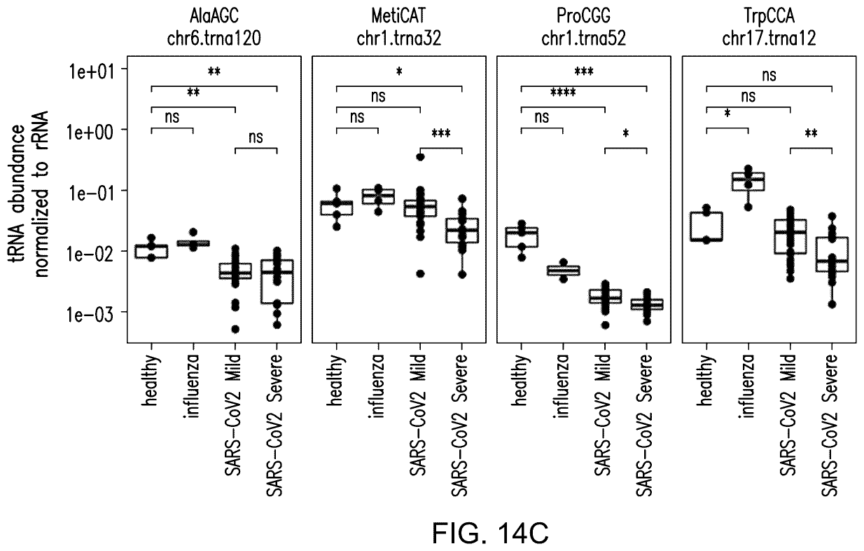

tRNA fragmentation occured extensively in all samples. Fractions of reads mapped to sequential regions along tRNA are shown for healthy control (n=5), influenza infected (n=4), and SARS-CoV-2 infected (n=57) individuals (FIG. 14A). Fragmentation of tRNAs shows consistent and unique patterns for each patient group. Fragment cleavage occurs mostly in the anticodon region.

Fragmentation of specific tRNAs can distinguish uninfected, influenza and SARS-CoV-2 infected individuals (FIG. 14B); ns, not significant, P-values: * <0.05; ** <0.01; *** <10⁻³, **** <10⁻⁴. Abundance differences of specific full length tRNAs, normalized to 5.8S rRNA, can distinguish different viral infections (i.e., influenza from SARS Cov-2), and even distinguish between SARS-CoV-2 patients who went on to develop mild (n=36) or severe (n=21) symptoms (FIG. 14C). Patients who developed mild symptoms show a higher portion of fragmented tRNAs, consistent with greater RNase secretion from a robust innate immune response.

Another parameter examined in the same sequencing data is quantitative comparison of RNA modifications through RT mutation signatures. Specific tRNA modifications could distinguish healthy patients from either viral infection and SARS-CoV-2 infection symptom development (FIG. 14D).

The results demonstrate that RNA-seq technology is capable of generating high quality tRNA sequencing results from banked nasopharyngeal swaps. tRNA fragmentation profiles in the human nasopharyngeal region have the potential to be biomarkers as prognostics for infection outcomes by identification of patients at high risk for complication from respiratory virus infection.

### EXAMPLE 3

### Colorectal Cancer

The following Example demonstrates use of the RNA-seq method of RNA library preparation, which is generally described in Example 1 and uses a hairpin oligonucleotide as described herein, for the development of a potential colorectal cancer (CRC) biomarker, in accordance with aspects of the invention

tRNA from tumor and adjacent tissues from 6 patients with CRC were sequenced. The experiment explored the feasibility of studying tRNA from these samples, and determined whether tumors are homogeneous or exhibit tRNA-level variations related to patient demographics (i.e., body mass index, BMI).

The majority of the RNA data obtained from these samples was tRNA (71%), as expected. The remainder of the RNA was rRNA (7.3%), mt_tRNA (2.7%) and other RNAs (19%).

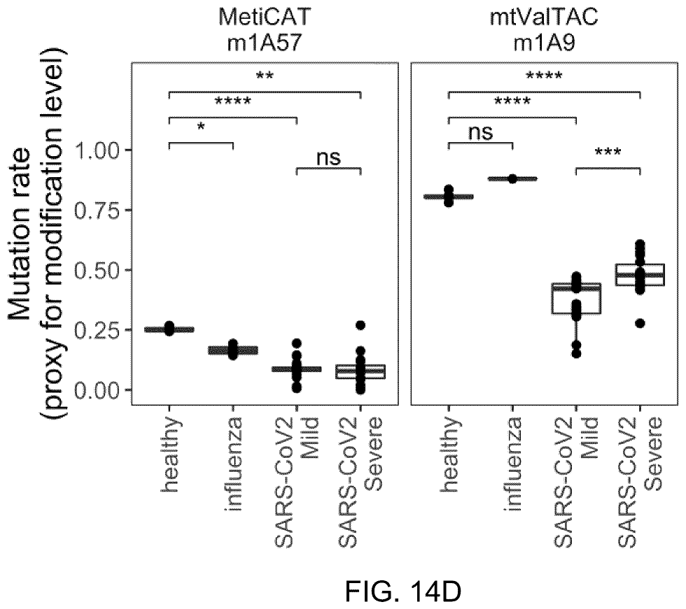

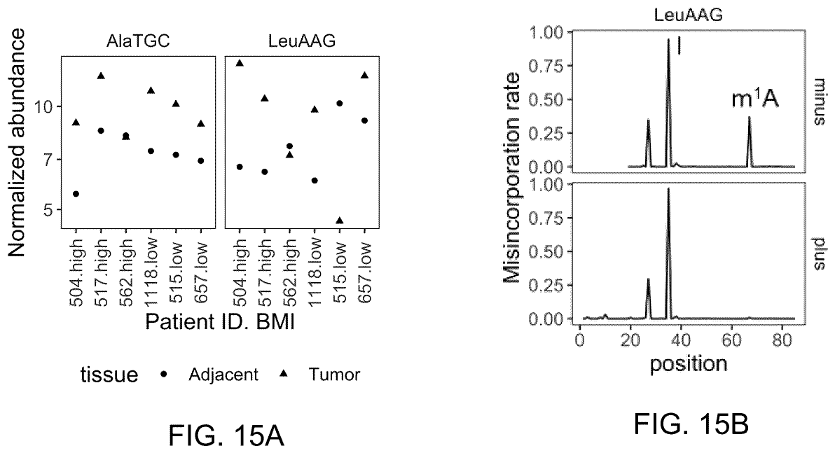

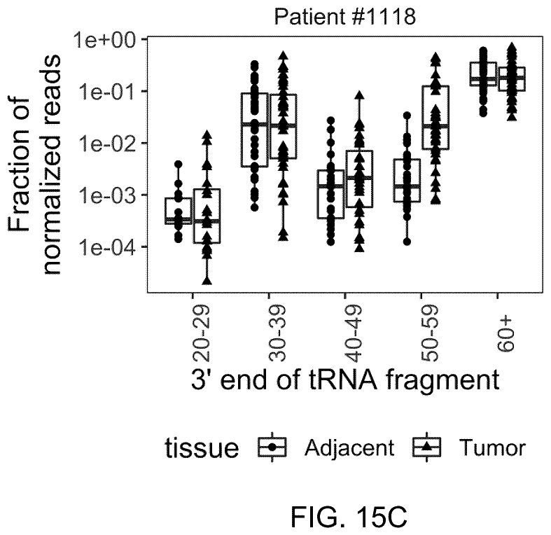

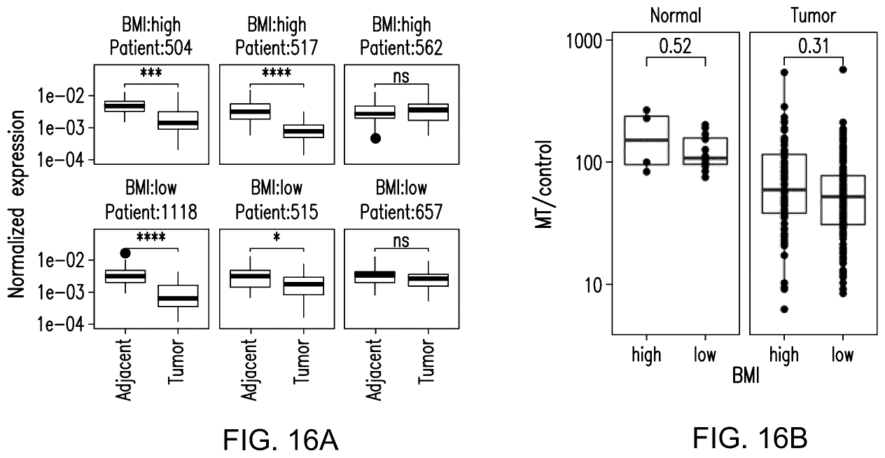

High-resolution data enabled the examination of different properties for >300 chromosomal-encoded tRNA genes (FIG. 15) and 22 mitochondrial-encoded tRNA genes (FIG. 16).

FIG. 15 depicts measures of tRNA-seq abundance, modification, and fragmentation in tumor and adjacent tissues from 6 patients with colorectal cancer (CRC). Expression level (FIG. 15A): tRNA abundance reveals significant heterogeneity among patients. For example, expression of tRNAs that read codons of amino acid alanine is relatively constant among patients, with tumors expressing ˜2-fold higher levels than adjacent tissue (left panel). By contrast, tRNAs that read codons of amino acid leucine show distinct expression patterns in each patient, regardless of BMI or tRNA^{Ala} expression level (right panel). Modification (FIG. 15B): tRNA-seq detected post-transcriptional methylation modifications resulting in nucleotide misincorporations during sequencing library construction (upper panel). Certain modifications were validated by treating samples with demethylating enzymes that remove methylations, thereby abolishing misincorporation (m¹A), while different a modification (1) was unaffected (lower panel). Fragmentation (FIG. 15C): tRNA fragments are produced by cellular nuclease cleavage in response to different cellular conditions, and belong to their own family of regulatory non-coding RNAs. RNA-seq analysis distinguishes among tRNAs with different 3′ ends, which can be grouped based on the location of the cleavage sites in tRNA secondary structure regions (e.g., D-loop, anticodon-loop, T-loop). As expected, tRNA fragments account for ˜1-10% of total tRNA reads, with cleavage in the anticodon-loop (30-39) the most common. Unexpectedly, cleavage in the T loop (50-59) is markedly different between tumor and adjacent tissue, suggesting that tRNA fragment profiles could be useful biomarkers.

FIG. 16 depicts tumor expression patterns of mitochondrial tRNAs in individual patients. Mitochondrial tRNAs are significantly under-expressed in tumors compared to adjacent tissue for 4 out of 6 patients (FIG. 16A), a finding consistent with the Warburg effect and mitochondrial dysfunction in cancers. In these samples, there was not a strong pattern of difference between samples from patients with low and high BMI. When the analysis was extended to include hundreds of samples in The Cancer Genome Atlas (TCGA), the data show that expression of mitochondrial genes is significantly lower in tumors from patients with low BMI compared to those with high BMI (FIG. 16B).

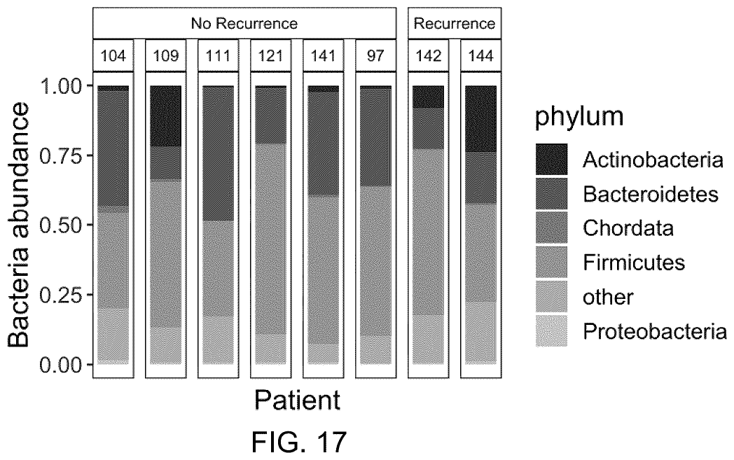

In addition to tRNA, the RNA-seq technology also captured small RNAs from microbes, enabling the use of microbial 5S rRNA to analyze the compositions of microbial communities in individual patients (FIG. 17). Three of the patients show high fractions of actinobacteria. Two of the three patients are known to have developed recurrence of CRC; the study is being extended to see if the CRC status of the third patient changes.

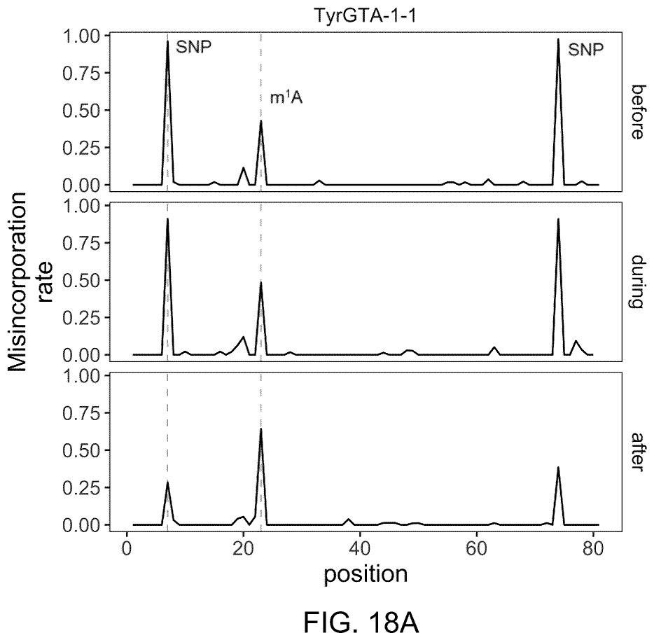

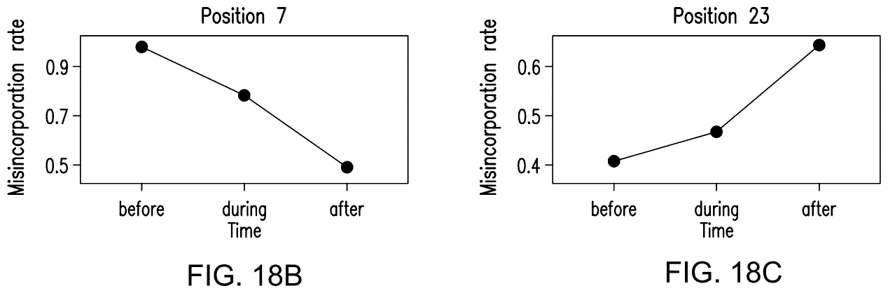

Chromosomal tRNA results in an individual patient can also be used to identify species differences through base modifications and inter-species polymorphisms at high resolution. Initial analysis focused on the commensal gut bacteria E. faecalis, which are known to be associated with CRC recurrence. Misincorporation can be due to tRNA base modifications (m1A) or base diversity (SNP) reflecting genetic diversity in the microbiome sample. Misincorporation results along tRNA^{Tyr} from E. faecalis in samples taken from a patient before, during and after surgery (FIG. 18A) provide several insights. First, positions 7 and 74 show changes in misincorporation over time. Based on the established knowledge of tRNA structure and modification, and the mutations' insensitivity to demethylase treatment, these changes were identified as representing genetic diversity due to differential accumulation of closely-related species of the enterococcus genus after surgery (FIG. 18B). The decreasing diversity seen is indicative of significantly altered species composition after surgery. In contrast, misincorporation at position 23 was sensitive to demethylase treatment, indicating that it results from a base modification in the tRNA, in this case N1-methyladenosine (FIG. 18C). The fraction of misincorporation increased by ˜20% after surgery, suggesting that this modification in the gut enterococcus reflects effects of treatment status.

The results demonstrate that analyses enabled by RNA-seq technology permit many different insights into RNA variability in tumors.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred aspects of this invention are described herein, including the best mode known to the inventors for carrying out the invention. Variations of those preferred aspects may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A hairpin oligonucleotide comprising a 3′-terminal nucleotide, wherein the sugar component of the 3′-terminal nucleotide comprises a 2′-hydroxyl and a 3′-phosphate.

2. The hairpin oligonucleotide of claim 1, wherein the sugar component of the 3′-terminal nucleotide is a pentose and the pentose is ribose.

3. A hairpin oligonucleotide comprising a 3′-terminal nucleotide wherein the sugar position of the 3′-terminal nucleotide comprises a 2′,3′-dialdehyde oxidation product of a sugar.

4. The hairpin oligonucleotide of any one of claims 1-3, further comprising a 5′-terminal ribonucleotide.

5. The hairpin oligonucleotide of any one of claims 1-4, further comprising:
(a) a barcode sequence,
(b) an affinity moiety tagged-nucleotide internal to the loop of the hairpin, and
(c) a primer binding site.

6. The hairpin nucleotide of claim 5, comprising the sequence:
5′-Phos-rA CT-X-AGA TCG GAA GAG CAC
ACG AT (SEQ ID NO: 86)-LT-AGA CGT GTG
CTC TTC CGA TCT (SEQ ID NO: 87)-Z-AG
rU-3′-Phos,
wherein X is a barcode of at least 3, 4, 5 or 6 nucleotides, LT is an affinity moiety tagged-Thymine nucleotide, and Z is a sequence of nucleotides that is the reverse complement of the barcode sequence.

7. The hairpin nucleotide of claim 5, comprising the sequence:
5′-Phos-rA CT-X-GAT CGT CGG ACT GTA
GAA CAT (SEQ ID NO: 88)-LT-AG AGT TCT
ACA GTC CGA CGA TC (SEQ ID NO: 89)-
Z-AG rU-3′-Phos,
wherein X is a barcode of at least 3, 4, 5 or 6 nucleotides, LT is an affinity moiety tagged-Thymine nucleotide, and Z is a sequence of nucleotides that is the reverse complement of the barcode sequence.

8. The hairpin oligonucleotide of any one of claims 1-7 immobilized on a solid support.

9. Use of the hairpin oligonucleotide of any one of claims 1-8 in preparing an RNA-sequence library.

10. Use of the hairpin oligonucleotide of any one of claims 1-8 in a multiplex method of preparing an RNA-sequence library.

11. Use of the hairpin oligonucleotide of any one of claims 1-8 in developing a biomarker.

12. The use of claim 11, wherein the biomarker is developed from liquid biopsy.

13. The use of claim 11 or 12, wherein developing the biomarker comprises generating a tRNA fragmentation profile.

14. The use of the hairpin oligonucleotide of any one of claims 1-8 in developing a biomarker for viral disease severity.

15. Use of the hairpin oligonucleotide of any one of claims 1-8 in developing a biomarker for cancer.

16. A solid support comprising a ligand moiety and a hairpin oligonucleotide, the oligonucleotide comprising an affinity moiety and a 3′-terminal nucleotide, wherein the sugar component of the 3′-terminal nucleotide comprises a 2′ hydroxyl and a 3′ phosphate,
and wherein the oligonucleotide is immobilized on the solid support through binding of the affinity moiety of the hairpin oligonucleotide to the ligand moiety of the solid support.

17. The solid support of claim 16, wherein the affinity moiety is biotin and the ligand moiety is streptavidin.

18. The solid support of claim 16 or 17, wherein the solid support is a bead.

19. The solid support of any one of claims 16-18, wherein the oligonucleotide further comprises:
(a) a 5′-terminal nucleotide as a ribonucleotide, hairpin, and
(b) a barcode sequence,
(c) a nucleotide tagged with the affinity moiety internal to the loop of the
(d) a primer binding site.

20. Use of the solid support of any one of claims 16-19 in preparing an RNA-sequence library.

21. Use of the solid support of any one of claims 16-19 in a multiplex method of preparing an RNA-sequence library.

22. A method of preparing an RNA-sequence library comprising:
(a) ligating an RNA sequence to a hairpin oligonucleotide to form a construct, the oligonucleotide comprising a 3′-terminal nucleotide, wherein the sugar component of the 3′-terminal nucleotide comprises a 2′ hydroxyl and a 3′ phosphate,
(b) reverse-transcribing the RNA sequence as a cDNA sequence, and
(c) amplifying the cDNA sequence using PCR.

23. The method of claim 22, wherein the hairpin oligonucleotide further comprises:
(i) a 5′-terminal nucleotide as a ribonucleotide,
(ii) a barcode sequence,
(iii) an affinity moiety-tagged nucleotide internal to the loop of the hairpin, and
(iv) a primer binding site.

24. The method of claim 22 or claim 23, further comprising dephosphorylating the 3′-phosphate after ligation and oxidizing 3′-terminal nucleotides comprising a 2′,3′-diol with periodate after reverse transcription.

25. The method of claim 24, further comprising demethylating Watson-Crick face methylations on nucleotides of the RNA sequence after ligation and before dephosphorylation.

26. The method of any one of claims 22-25, further comprising digesting the RNA sequence after reverse transcription and performing a second ligation to add a second primer binding site before amplification.

27. The method of claim 22 or claim 23, further comprising immobilizing the construct on a solid support after ligation.

28. The method of claim 27, further comprising dephosphorylating the 3′-phosphate after immobilization and oxidizing 3′-terminal nucleotides comprising a 2′,3′-diol with periodate after reverse transcription.

29. The method of claim 28, further comprising demethylating Watson-Crick face methylations on nucleotides of the RNA sequence after immobilization and before dephosphorylation.

30. The method of any one of claims 27-29, further comprising digesting the RNA sequence after reverse transcription and performing a second ligation to add a second primer binding site before amplification.

31. The method of any one of claims 22-30, wherein the RNA sequence comprises total RNA, small RNAs, tRNAs, micro RNAs, piRNAs, or any combination thereof.

32. The method of any one of claims 22-31, wherein the method comprises a multiplex method.


SEQUENCE LISTING (89 sequences)

SEQ ID NO: 1 - DNA, 56 bp, Artificial Sequence
  Synthetic
actggaaagatcggaagagcacacgattagacgtgtgctcttccgatctttccagu

SEQ ID NO: 2 - DNA, 59 bp, Artificial Sequence
  Synthetic
ccaactggaaagatcggaagagcacacgattagacgtgtgctcttccgatctttccagu

SEQ ID NO: 3 - DNA, 62 bp, Artificial Sequence
  Synethic
ccaactggaaagatcggaagagcacacgattagacgtgtgctcttccgatctttccagutgg

SEQ ID NO: 4 - DNA, 59 bp, Artificial Sequence
  Synthetic
actggaaagatcggaagagcacacgattagacgtgtgctcttccgatctttccagutgg

SEQ ID NO: 5 - DNA, 35 bp, Artificial Sequence
  Synthetic
  misc_feature (1)..(6): n is a, c, g, or t
nnnnnngatcgtcggactgtagaactctgaacgtc

SEQ ID NO: 6 - DNA, 28 bp, Artificial Sequence
  Synthetic
acgttcagagttctacagtccgacgatc

SEQ ID NO: 7 - DNA, 21 bp, Artificial Sequence
  Synthetic
agacgtgtgctcttccgatct

SEQ ID NO: 8 - RNA, 13 nt, Prevotella melaninogenica
aagacgggcaagu

SEQ ID NO: 9 - RNA, 18 nt, Prevotella melaninogenica
gcuuguccaccccgccuc

SEQ ID NO: 10 - DNA, 12 bp, Prevotella melaninogenica
ccaccccgcctc

SEQ ID NO: 11 - RNA, 14 nt, Rothia mucilaginosa
ucuugccucuuacg

SEQ ID NO: 12 - RNA, 17 nt, Rothia mucilaginosa
cguaagggguuuuucua

SEQ ID NO: 13 - DNA, 10 bp, Rothia mucilaginosa
ggtttttcta

SEQ ID NO: 14 - DNA, 54 bp, Artificial Sequence
  Synthetic
  misc_feature (4)..(6): n is a, c, g, t or u
  misc_feature (49)..(51): n is a, c, g, t or u
actnnnagatcggaagagcacacgattagacgtgtgctcttccgatctnnnagu

SEQ ID NO: 15 - DNA, 56 bp, Artificial Sequence
  Synthetic
  misc_feature (4)..(7): n is a, c, g, t or u
  misc_feature (50)..(53): n is a, c, g, t or u
actnnnnagatcggaagagcacacgattagacgtgtgctcttccgatctnnnnagu

SEQ ID NO: 16 - DNA, 58 bp, Artificial Sequence
  Synthetic
  misc_feature (4)..(8): n is a, c, g, t or u
  misc_feature (51)..(55): n is a, c, g, t or u
actnnnnnagatcggaagagcacacgattagacgtgtgctcttccgatctnnnnnagu

SEQ ID NO: 17 - DNA, 60 bp, Artificial Sequence
  Synthetic
  misc_feature (4)..(9): n is a, c, g, t or u
  misc_feature (52)..(57): n is a, c, g, t or u
actnnnnnnagatcggaagagcacacgattagacgtgtgctcttccgatctnnnnnnagu

SEQ ID NO: 18 - DNA, 56 bp, Artificial Sequence
  Synthetic
  misc_feature (4)..(6): n is a, c, g, t or u
  misc_feature (51)..(53): n is a, c, g, t or u
actnnngatcgtcggactgtagaacattagagttctacagtccgacgatcnnnagu

SEQ ID NO: 19 - DNA, 58 bp, Artificial Sequence
  Synthetic
  misc_feature (4)..(7): n is a, c, g, t or u
  misc_feature (52)..(55): n is a, c, g, t or u
actnnnngatcgtcggactgtagaacattagagttctacagtccgacgatcnnnnagu

SEQ ID NO: 20 - DNA, 60 bp, Artificial Sequence
  Synthetic
  misc_feature (4)..(8): n is a, c, g, t or u
  misc_feature (53)..(57): n is a, c, g, t or u
actnnnnngatcgtcggactgtagaacattagagttctacagtccgacgatcnnnnnagu

SEQ ID NO: 21 - DNA, 62 bp, Artificial Sequence
  Synthetic
  misc_feature (4)..(9): n is a, c, g, t or u
  misc_feature (54)..(59): n is a, c, g, t or u
actnnnnnngatcgtcggactgtagaacattagagttctacagtccgacgatcnnnnnnagu

SEQ ID NO: 22 - DNA, 25 bp, Artificial Sequence
  Synthetic
  misc_feature (1)..(6): n is a, c, g, or t
nnnnnngatcgtcggactgtagaac

SEQ ID NO: 23 - DNA, 25 bp, Artificial Sequence
  Synthetic
  misc_feature (1)..(6): n is a, c, g, or t
nnnnnnagatcggaagagcacacgc

SEQ ID NO: 24 - DNA, 56 bp, Artificial Sequence
  Synthetic
acttgaaagatcggaagagcacacgattagacgtgtgctcttccgatctttcaagu

SEQ ID NO: 25 - DNA, 56 bp, Artificial Sequence
  Synthetic
acttaggagatcggaagagcacacgattagacgtgtgctcttccgatctcctaagu

SEQ ID NO: 26 - DNA, 56 bp, Artificial Sequence
  Synthetic
acttgttagatcggaagagcacacgattagacgtgtgctcttccgatctaacaagu

SEQ ID NO: 27 - DNA, 56 bp, Artificial Sequence
  Synthetic
acttaccagatcggaagagcacacgattagacgtgtgctcttccgatctggtaagu

SEQ ID NO: 28 - DNA, 56 bp, Artificial Sequence
  Synthetic
acttccaagatcggaagagcacacgattagacgtgtgctcttccgatcttggaagu

SEQ ID NO: 29 - DNA, 56 bp, Artificial Sequence
  Synthetic
acttttgagatcggaagagcacacgattagacgtgtgctcttccgatctcaaaagu

SEQ ID NO: 30 - DNA, 56 bp, Artificial Sequence
  Synthetic
actttgtagatcggaagagcacacgattagacgtgtgctcttccgatctacaaagu

SEQ ID NO: 31 - DNA, 56 bp, Artificial Sequence
  Synthetic
acttcacagatcggaagagcacacgattagacgtgtgctcttccgatctgtgaagu

SEQ ID NO: 32 - DNA, 56 bp, Artificial Sequence
  Synthetic
acttgcgagatcggaagagcacacgattagacgtgtgctcttccgatctcgcaagu

SEQ ID NO: 33 - DNA, 56 bp, Artificial Sequence
  Synthetic
acttctaagatcggaagagcacacgattagacgtgtgctcttccgatcttagaagu

SEQ ID NO: 34 - DNA, 56 bp, Artificial Sequence
  Synthetic
acttattagatcggaagagcacacgattagacgtgtgctcttccgatctaataagu

SEQ ID NO: 35 - DNA, 56 bp, Artificial Sequence
  Synthetic
acttggcagatcggaagagcacacgattagacgtgtgctcttccgatctgccaagu

SEQ ID NO: 36 - DNA, 58 bp, Artificial Sequence
  Synthetic
actggaagatcgtcggactgtagaacattagagttctacagtccgacgatcttccagu

SEQ ID NO: 37 - DNA, 58 bp, Artificial Sequence
  Synthetic
actcagagatcgtcggactgtagaacattagagttctacagtccgacgatctctgagu

SEQ ID NO: 38 - DNA, 58 bp, Artificial Sequence
  Synthetic
actaccagatcgtcggactgtagaacattagagttctacagtccgacgatctggtagu

SEQ ID NO: 39 - DNA, 58 bp, Artificial Sequence
  Synthetic
acttcaggatcgtcggactgtagaacattagagttctacagtccgacgatcctgaagu

SEQ ID NO: 40 - DNA, 58 bp, Artificial Sequence
  Synthetic
actatgggatcgtcggactgtagaacattagagttctacagtccgacgatcccatagu

SEQ ID NO: 41 - DNA, 58 bp, Artificial Sequence
  Synthetic
actgatggatcgtcggactgtagaacattagagttctacagtccgacgatccatcagu

SEQ ID NO: 42 - DNA, 58 bp, Artificial Sequence
  Synthetic
actctacgatcgtcggactgtagaacattagagttctacagtccgacgatcgtagagu

SEQ ID NO: 43 - DNA, 58 bp, Artificial Sequence
  Synthetic
acttaccgatcgtcggactgtagaacattagagttctacagtccgacgatcggtaagu

SEQ ID NO: 44 - DNA, 57 bp, Artificial Sequence
  Synthetic
actagtcatcgtcggactgtagaacattagagttctacagtccgacgatcgactagu

SEQ ID NO: 45 - DNA, 58 bp, Artificial Sequence
  Synthetic
acttggtgatcgtcggactgtagaacattagagttctacagtccgacgatcaccaagu

SEQ ID NO: 46 - DNA, 58 bp, Artificial Sequence
  Synthetic
actgtctgatcgtcggactgtagaacattagagttctacagtccgacgatcagacagu

SEQ ID NO: 47 - DNA, 58 bp, Artificial Sequence
  Synthetic
actccttgatcgtcggactgtagaacattagagttctacagtccgacgatcaaggagu

SEQ ID NO: 48 - DNA, 56 bp, Artificial Sequence; identical to SEQ ID NO: 1
  Synthetic

SEQ ID NO: 49 - DNA, 56 bp, Artificial Sequence
  Synthetic
actcagaagatcggaagagcacacgattagacgtgtgctcttccgatcttctgagu

SEQ ID NO: 50 - DNA, 56 bp, Artificial Sequence
  Synthetic
actaccaagatcggaagagcacacgattagacgtgtgctcttccgatcttggtagu

SEQ ID NO: 51 - DNA, 56 bp, Artificial Sequence
  Synthetic
acttcagagatcggaagagcacacgattagacgtgtgctcttccgatctctgaagu

SEQ ID NO: 52 - DNA, 56 bp, Artificial Sequence
  Synthetic
actatggagatcggaagagcacacgattagacgtgtgctcttccgatctccatagu

SEQ ID NO: 53 - DNA, 56 bp, Artificial Sequence
  Synthetic
actgatgagatcggaagagcacacgattagacgtgtgctcttccgatctcatcagu

SEQ ID NO: 54 - DNA, 56 bp, Artificial Sequence; identical to SEQ ID NO: 52
  Synthetic

SEQ ID NO: 55 - DNA, 56 bp, Artificial Sequence; identical to SEQ ID NO: 27
  Synthetic

SEQ ID NO: 56 - DNA, 56 bp, Artificial Sequence
  Synthetic
actagtcagatcggaagagcacacgattagacgtgtgctcttccgatctgactagu

SEQ ID NO: 57 - DNA, 56 bp, Artificial Sequence
  Synthetic
acttggtagatcggaagagcacacgattagacgtgtgctcttccgatctaccaagu

SEQ ID NO: 58 - DNA, 56 bp, Artificial Sequence
  Synthetic
actgtctagatcggaagagcacacgattagacgtgtgctcttccgatctagacagu

SEQ ID NO: 59 - DNA, 56 bp, Artificial Sequence
  Synthetic
actccttagatcggaagagcacacgattagacgtgtgctcttccgatctaaggagu

SEQ ID NO: 60 - DNA, 56 bp, Artificial Sequence; identical to SEQ ID NO: 1
  Synthetic

SEQ ID NO: 61 - DNA, 54 bp, Artificial Sequence
  Synthetic
aatgaacggcgaccaccgagatctacacgttcagagttctacagtccgacgatc

SEQ ID NO: 62 - DNA, 64 bp, Artificial Sequence
  Synthetic
caagcagaagacggcatacgagatcgtgatgtgactggagttcagacgtgtgctcttccgatct

SEQ ID NO: 63 - DNA, 64 bp, Artificial Sequence
  Synthetic
caagcagaagacggcatacgagatacatcggtgactggagttcagacgtgtgctcttccgatct

SEQ ID NO: 64 - DNA, 64 bp, Artificial Sequence
  Synthetic
caagcagaagacggcatacgagatgcctaagtgactggagttcagacgtgtgctcttccgatct

SEQ ID NO: 65 - DNA, 64 bp, Artificial Sequence
  Synthetic
caagcagaagacggcatacgagattggtcagtgactggagttcagacgtgtgctcttccgatct

SEQ ID NO: 66 - DNA, 64 bp, Artificial Sequence
  Synthetic
caagcagaagacggcatacgagatcactgtgtgactggagttcagacgtgtgctcttccgatct

SEQ ID NO: 67 - DNA, 64 bp, Artificial Sequence
  Synthetic
caagcagaagacggcatacgagatattggcgtgactggagttcagacgtgtgctcttccgatct

SEQ ID NO: 68 - DNA, 64 bp, Artificial Sequence
  Synthetic
caagcagaagacggcatacgagatgatctggtgactggagttcagacgtgtgctcttccgatct

SEQ ID NO: 69 - DNA, 64 bp, Artificial Sequence
  Synthetic
caagcagaagacggcatacgagattcaagtgtgactggagttcagacgtgtgctcttccgatct

SEQ ID NO: 70 - DNA, 64 bp, Artificial Sequence
  Synthetic
caagcagaagacggcatacgagatctgatcgtgactggagttcagacgtgtgctcttccgatct

SEQ ID NO: 71 - DNA, 64 bp, Artificial Sequence
  Synthetic
caagcagaagacggcatacgagataagctagtgactggagttcagacgtgtgctcttccgatct

SEQ ID NO: 72 - DNA, 64 bp, Artificial Sequence
  Synthetic
caagcagaagacggcatacgagatgtagccgtgactggagttcagacgtgtgctcttccgatct

SEQ ID NO: 73 - DNA, 64 bp, Artificial Sequence
  Synthetic
caagcagaagacggcatacgagattacaaggtgactggagttcagacgtgtgctcttccgatct

SEQ ID NO: 74 - DNA, 64 bp, Artificial Sequence
  Synthetic
caagcagaagacggcatacgagatttgactgtgactggagttcagacgtgtgctcttccgatct

SEQ ID NO: 75 - DNA, 64 bp, Artificial Sequence
  Synthetic
caagcagaagacggcatacgagatggaactgtgactggagttcagacgtgtgctcttccgatct

SEQ ID NO: 76 - DNA, 64 bp, Artificial Sequence
  Synthetic
caagcagaagacggcatacgagattgacatgtgactggagttcagacgtgtgctcttccgatct

SEQ ID NO: 77 - DNA, 64 bp, Artificial Sequence
  Synthetic
caagcagaagacggcatacgagatggacgggtgactggagttcagacgtgtgctcttccgatct

SEQ ID NO: 78 - DNA, 64 bp, Artificial Sequence
  Synthetic
caagcagaagacggcatacgagatctctacgtgactggagttcagacgtgtgctcttccgatct

SEQ ID NO: 79 - DNA, 64 bp, Artificial Sequence
  Synthetic
caagcagaagacggcatacgagatgcggacgtgactggagttcagacgtgtgctcttccgatct

SEQ ID NO: 80 - DNA, 64 bp, Artificial Sequence
  Synthetic
caagcagaagacggcatacgagattttcacgtgactggagttcagacgtgtgctcttccgatct

SEQ ID NO: 81 - DNA, 64 bp, Artificial Sequence
  Synthetic
caagcagaagacggcatacgagatggccacgtgactggagttcagacgtgtgctcttccgatct

SEQ ID NO: 82 - DNA, 64 bp, Artificial Sequence
  Synthetic
caagcagaagacggcatacgagatcgaaacgtgactggagttcagacgtgtgctcttccgatct

SEQ ID NO: 83 - DNA, 64 bp, Artificial Sequence
  Synthetic
caagcagaagacggcatacgagatcgtacggtgactggagttcagacgtgtgctcttccgatct

SEQ ID NO: 84 - DNA, 64 bp, Artificial Sequence
  Synthetic
caagcagaagacggcatacgagatccactcgtgactggagttcagacgtgtgctcttccgatct

SEQ ID NO: 85 - DNA, 64 bp, Artificial Sequence
  Synthetic
caagcagaagacggcatacgagatgctaccgtgactggagttcagacgtgtgctcttccgatct

SEQ ID NO: 86 - DNA, 20 bp, Artificial Sequence
  Synthetic
agatcggaagagcacacgat

SEQ ID NO: 87 - DNA, 21 bp, Artificial Sequence
  Synthetic
agacgtgtgctcttccgatct

SEQ ID NO: 88 - DNA, 21 bp, Artificial Sequence
  Synthetic
gatcgtcggactgtagaacat

SEQ ID NO: 89 - DNA, 22 bp, Artificial Sequence
  Synthetic
agagttctacagtccgacgatc